(19) United States
(12) Patent Application Publication
(10) Pub. No.: US 2022/0168243 A1
(43) Pub. Date: Jun. 2, 2022

(54) CORONAVIRUS TREATMENT COMPOSITIONS AND METHODS

(71) Applicant: The Research Foundation for The State University of New York, Amherst, NY (US)

(72) Inventors: Ram Samudrala, Amherst, NY (US); Zackary Falls, Amherst, NY (US); William Mangione, Amherst, NY (US)

(21) Appl. No.: 17/539,791

(22) Filed: Dec. 1, 2021

Related U.S. Application Data
(60) Provisional application No. 63/120,633, filed on Dec. 2, 2020.

(51) Int. Cl. A61K 31/137; A61K 45/06

(52) U.S. Cl. CPC A61K 31/137; A61K 45/06

(57) ABSTRACT

Methods for the inhibition and antiviral treatment and prevention of coronavirus infections and particularly coronavirus antiviral therapies for the inhibition of coronavirus or treatment or prevention of coronavirus diseases in humans and animals by administering to a human or animal suffering from coronavirus infection, an effective amount of one or more specifically identified antiviral compounds that inhibit coronavirus infection or replication.

### CROSS-REFERENCE TO PRIORITY APPLICATION

This application claims priority to U.S. Provisional Application No. 63/120,633, filed Dec. 2, 2020, which is incorporated herein by reference in its entirety.

### STATEMENT REGARDING FEDERALLY FUNDED RESEARCH

This invention was made with government support under Grant Numbers LM012495, TR001412, and OD006779, awarded by the National Institutes of Health. The government has certain rights in the invention.

### FIELD

The present application relates to the field of viral inhibition and antiviral treatment methods and particularly relates to coronavirus antiviral therapies for the inhibition of coronavirus or treatment of coronavirus diseases in humans and animals.

### BACKGROUND

Coronaviruses are a group of related viruses that cause diseases in mammals and birds. In humans, coronaviruses cause respiratory tract infections that can be mild, such as some cases of the common cold, although most colds are caused by rhinoviruses. Other respiratory tract infections caused by coronaviruses that can be lethal to humans include Severe Acute Respiratory Syndrome (SARS), Middle East Respiratory Syndrome (MERS), and 2019 Novel Coronavirus (COVID-19), which is responsible for the pandemic of 2020. Other natural hosts include pig, dog, cat, mice, rats, cow, rabbit, chicken and turkey. Symptoms in other species vary: in chickens, they cause an upper respiratory tract disease, while in cows and pigs they cause diarrhea. Other diseases caused by coronavirus include infectious peritonitis, runting nephritis, pancreatitis parotitis and adenitis.

Coronaviruses are large, enveloped, single-stranded RNA viruses having the largest genomes of all RNA viruses. They replicate by a unique mechanism that results in a high frequency of recombination.

Identification of coronaviruses is simplified by the characteristic appearance of a crown, or corona, around the virions (virus particles), when viewed under two-dimensional transmission electron microscopy, due to the surface of the virus particle being covered in well-separated, petal-shaped glycoprotein peplomers or spikes, having a diameter of 80-160 nm, that project from the virions. The spike (S, also known as E2) glycoprotein is a Class I viral fusion protein located on an outer envelope of the virion and plays a critical role in viral infection by recognizing host cell receptors. The spike (S) glycoprotein is therefore responsible for host cell attachment and for mediating host cell membrane and viral membrane fusion during infection.

Many molecules of a basic phosphoprotein, N(50-60K), encapsidate the genomic RNA to form a long, flexible nucleocapsid having helical symmetry. In thin sections of virions, these helical nucleocapsids appear as tubular strands. The nucleocapsid lies within a lipoprotein envelope, which is a bilayer containing two viral glycoproteins, the matrix glycoprotein (M, also known as E1) and the spike glycoprotein (S, or E2), mentioned above. Only a small anchor domain of the S glycoprotein is embedded in the lipid bilayer. The envelopes of all coronaviruses are lipid bilayers containing the M and S glycoproteins. Some coronaviruses also include a third envelope protein, Hemagglutinin-esterase glycoprotein (HE, also known as E3).

Matrix, or M glycoprotein determines budding from endoplasmic reticulum and Golgi membranes, forms the viral envelope and interacts with the viral nucleocapsid. Spike, or S glycoprotein binds to host-cell receptor glycoprotein, induces cell fusion, may induce fusion of viral envelope with cell membrane, induces neutralizing antibody and elicits cell-mediated immunity. Hemagglutinin-esterase, or HE glycoprotein binds to 9-O-acetylated neuramic acid residues on cell membranes, causes hemagglutination and cleaves acetyl group of 9-O-acetylated nuraminic acid.

The nucleocapsid is formed from multiple copies of the nucleocapsid (N) protein, which are bound to the positive-sense single-stranded RNA genome in a continuous beads-on-α-string type conformation. The lipid bilayer envelope, membrane proteins, and nucleocapsid protect the virus when it is outside the host cell.

Because coronaviruses exhibit strong species and tissue specificity, most coronaviruses naturally infect only one species or several closely related species. However, transmission from infected animal species to humans has occurred, resulting in novel virus infections to which the human immune system has no innate immunity. Fortunately, virus replication is often limited to epithelial cells of the respiratory or enteric tracts and macrophages.

Human to human transmission of coronaviruses is primarily thought to occur among close contacts via respiratory droplets generated by sneezing and coughing. Infection begins when the virus enters the host organism and the spike protein attaches to its complementary host cell receptor. After attachment, a protease of the host cell cleaves and activates the receptor-attached spike protein. Depending on the host cell protease available, cleavage and activation allows cell entry through endocytosis or direct fusion of the viral envelope with the host membrane.

On entry into the host cell, the virus particle becomes uncoated, and its genome enters the cell cytoplasm. The coronavirus RNA genome has a 5′ methylated cap and a 3′ polyadenylated tail, which allows the RNA to attach to the host cell's ribosomes for translation. The host ribosome translates the initial overlapping open reading frame of the virus genome and forms a long polyprotein having its own proteases that cleave the polyprotein into multiple nonstructural proteins.

A number of the nonstructural proteins coalesce to form a multi-protein replicase-transcriptase complex (RTC). The main replicase-transcriptase protein is the RNA-dependent RNA polymerase (RdRP, also known as RNA replicase), which is directly involved in the replication and transcription of RNA from an RNA strand. The other nonstructural proteins in the complex assist in the replication and transcription process. For example, the exoribonuclease non-structural protein provides extra fidelity to replication by providing a proofreading function that the RNA-dependent RNA polymerase lacks.

One of the main functions of the replication/transcription complex (RTC) is to replicate the viral genome. Many nonstructural proteins (nsps) of the coronavirus are required for RTC function. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense messenger RNAs (mRNAs).

The replicated positive-sense genomic RNA becomes the genome of the progeny viruses. The mRNAs are gene transcripts of the last third of the virus genome after the initial overlapping reading frame. These mRNAs are translated by the host's ribosomes into the structural proteins and a number of accessory proteins. RNA translation occurs inside the endoplasmic reticulum (ER). The viral structural proteins S, E, and M move along the secretory pathway into the Golgi intermediate compartment. There, the M proteins direct most protein-protein interactions required for assembly of viruses following its binding to the nucleocapsid. Progeny viruses are then released from the host cell by exocytosis through secretory vesicles.

Coronaviruses vary significantly in risk factor. Some can kill more than 30% of those infected, such as MERS, while others, like the common cold, are annoying but relatively harmless. Coronaviruses cause colds with major symptoms, such as fever, and sore throat, mainly in the winter and early spring. However, some coronaviruses cause illnesses having more severe symptoms such as fever, dry cough, headache, muscle aches and difficulty breathing, and may result in the development of bronchitis and pneumonia, either direct viral bronchitis or pneumonia or secondary bacterial bronchitis or pneumonia. The human coronavirus discovered in 2003, known as SARS-CoV caused both upper and lower respiratory tract infections and resulted in the deaths of 774 humans.

The following strains of human coronaviruses are currently known: Human coronavirus 229E (HCoV-229E); Human coronavirus OC43 (HCoV-OC43); Severe acute respiratory syndrome coronavirus (SARS-CoV); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019”. Mutant strains of SARS-CoV-2 are also prevalent, including the Delta variant (B.1.617.2 variant) and the Lambda variant (C.37 variant). The coronaviruses HCoV-229E, -NL63, -OC43, and -HKU1 continually circulate in the human population and cause respiratory infections in adults and children world-wide.

In December 2019, a pneumonia outbreak was reported in Wuhan, China. Within the month, the disease was traced to a novel strain of coronavirus, named 2019-nCoV by the World Health Organization (WHO), also referred to as the virus causing the disease COVID-19, and later renamed SARS-CoV-2 by the International Committee on Taxonomy of Viruses. Within four months, over 7000 deaths and nearly 200,000 confirmed cases were reported in the pandemic. This strain of coronavirus has approximately 70% genetic similarity to the SARS-CoV, described above as causing over 700 human deaths, and 90% similarity to a bat coronavirus, so both viruses are widely suspected to originate from bats. The pandemic resulted in severe travel restrictions.

No fully effective antiviral drug exists for the treatment of SARS-CoV-2 infection.

Therefore, what is needed are improved methods for inhibiting coronavirus virus infection, thereby treating human and animal patients suffering from coronavirus infection.

### SUMMARY

Methods for inhibiting and preventing coronavirus infection, disrupting coronavirus replication and blocking coronavirus binding to target host cells or host cell receptors are described herein.

The methods of treating or preventing coronavirus infection in a human or animal involve administering to a human or animal suffering from coronavirus infection or exposed to coronavirus, an effective amount of one or more antiviral compounds identified herein that inhibit or prevent coronavirus infection or replication. Disruption of coronavirus replication is also achieved by combining a coronavirus organism with an effective amount of one or more of the antiviral compounds identified herein to inhibit replication. Blocking of coronavirus binding is achieved in the presence of one or more of the antiviral compounds identified, in an amount effective to prevent interaction of the coronavirus particle with the host cell or host cell receptor.

The compounds to be administered in accordance with the methods described herein block coronavirus glycoprotein binding to host cell receptors, via means such as the binding of the drug candidates to cell proteins of the host human or animal; or inhibit the function of coronavirus proteins, such as RdRP, thereby disrupting viral replication or cell entry. The feature being predicted is really the binding or “stickiness” of small molecules to viral proteins. This in turn is expected to discover inhibitors (of both host cell viral target proteins and pathogen proteins) which, in turn, would lead to clinical efficacy.

Lists of predictions were generated using three pipelines implemented in the Computational Analysis of Novel Drug Opportunities (CANDO) platform for shotgun drug discovery, repurposing, and design: 1) a homology-based approach using compounds with evidence of activity against the original SARS-CoV virus, the MERS virus, and SARS-CoV-2 virus; 2) a de novo method involving the top predicted binding affinities of approved drugs against structures from the COVID-19 proteome and 3) a method to find compounds similar to both remdesivir and favipiravir.

Compounds were identified as being putative drug repurposing candidates against SARS-CoV-2 if data demonstrated that they could inhibit the function of coronavirus NSP3-papain proteinase or if they could inhibit the function of coronavirus 3C-like proteinase, and thereby impede or block coronavirus replication.

The compositions are administered via any of several routes of administration, including orally, parenterally, intravenously, intraperitoneally, intracranially, intraspinally, intrathecally, intraventricularly, intramuscularly, subcutaneously, buccally, sublingually, intracavity, by inhalation or transdermally. Effective doses for any of the administration methods described herein can be extrapolated from dose-response curves derived from in vitro or animal model test systems.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below.

Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present document describes and refers to various embodiments of the invention. No particular embodiment is intended to define the scope of the invention. Rather, the embodiments merely provide non-limiting examples of various methods, that are at least included within the scope of the invention. Some embodiments of the present invention are summarized below, while others are described and shown elsewhere in the present document.

In one embodiment, a method is provided for treating or preventing coronavirus infection by administering to a human or animal infected with coronavirus, such as SARS-CoV-2 virus, an effective amount of one or more antiviral compounds, wherein the one or more antiviral compounds inhibit coronavirus binding to a host cell of the human or animal or disrupts replication of the coronavirus.

In another embodiment, a method is provided for treating or preventing coronavirus infection by administering to a human or animal infected with coronavirus, an effective amount of one or more antiviral compounds, wherein the one or more antiviral compounds inhibit coronavirus binding to a host cell of the human or animal or disrupts replication of the coronavirus wherein the one or more antiviral compound is Cinacalcet, Alverine, Alvimopan, Antazoline, Bepridil, Darifenacin, Desipramine, Diphenhydramine, Diphenidol, Flunarizine, Fosphenytoin, Furazolidone, Imipramine, Lifitegrast, Masitinib, Mifepristone, Nicardipine, Nilotinib, Olopatadine, Ospemifene, Oxprenolol, Perhexiline, Phenoxybenzamine, Phenylbutazone, Phenyltoloxamine, Pizotifen, Promazine, Stiripentol, Tamsulosin, Zolmitriptan, and salts thereof or combinations thereof.

In another embodiment, a method is provided for treating or preventing coronavirus infection by administering to a human or animal infected with coronavirus, an effective amount of one or more antiviral compounds, wherein the one or more antiviral compounds inhibit coronavirus binding to a host cell of the human or animal or disrupts replication of the coronavirus wherein the one or more antiviral compounds is one of the compounds identified below in Tables 1-3, alone or in combination with one or more additional compounds identified below in Tables 1-3.

### BRIEF DESCRIPTION OF THE DRAWINGS

FIG. 1 is a collection of graphs illustrating the results of the One-Step Assay, where SARS-CoV-2 was pretreated with each of 30 selected compounds described herein. The left y-axis defines the values for the bar-graph data plotting percent inhibition of SARS-CoV-2 in the presence of each of the 30 selected compounds where no values below zero (indicative of “enhancement”) are included. The right y-axis defines the percent change Relative Fluorescent Units (RFU) (as compared to media+drug only control) following 24 hour exposure (gray dashed line with diamonds) of drugs to cells.

FIG. 2 is a collection of graphs illustrating the results of the Two-Step Assay evaluating the overall ability of a compound to inhibit entry and replication of SARS-CoV-2 in cells when exposed to different concentrations of 30 selected compounds described herein. The left y-axis defines the values for the bar-graph data plotting percent inhibition of SARS-CoV-2 entry and replication in the presence of each of the selected compounds. The right y-axis expresses toxicity of the drug and defines the percent change Relative Fluorescent Units (RFU) following 24 hour (black dashed line with inverted triangles) and 48 hour (grey dotted line with open circles) exposure of drugs to cells.

FIG. 3 is a collection of graphs for a 4-fold dilution scheme starting at 100 μM concentration of each of the 30 selected compounds described herein. Following a 24-hour exposure period of Vero E6 cells to each of the 30 selected compounds, an assay was used to determine the toxicity of each of the 30 selected compounds under those conditions.

FIG. 4 is a collection of graphs for a 2-fold dilution scheme starting at 200 μM concentration of each of the 30 selected compounds described herein. Following a 24 hour exposure period of Vero E6 cells to each of the 30 selected compounds, an assay was used to determine the toxicity of each of the 30 selected compounds under those conditions.

FIG. 5 is a collection of graphs for a 2-fold dilution scheme starting at 200 μM concentration of each of the 30 selected compounds described herein. Following a 46-48 hour exposure period of Vero E6 cells to each of the 30 selected compounds, an assay was used to determine the toxicity of each of the 30 selected compounds under those conditions.

FIG. 6 is a collection of graphs for a 2-fold dilution scheme starting at 100 μM concentration of each of the 30 selected compounds described herein. Following a 46-48 hour exposure period of Vero E6 cells to each of the 30 selected compounds, an assay was used to determine the toxicity of each of the 30 selected compounds under those conditions.

### DETAILED DESCRIPTION

Provided herein are methods for the inhibition and prevention of coronavirus infection, disruption of coronavirus replication and blocking coronavirus binding to host. In particular, methods of treating coronavirus infection in a human or animal are provided by administering to a human or animal suffering from coronavirus infection, an effective amount of one or more antiviral compounds that inhibit coronavirus infection or replication. Disruption of coronavirus replication is achieved by combining a coronavirus organism with an effective amount of one or more of the antiviral compounds identified herein for a sufficient amount of time under appropriate conditions to inhibit replication. Blocking of coronavirus binding to host is achieved by combining a coronavirus organism and a host cell or host cell receptor to which the coronavirus organism will bind in the presence of an effective amount of one or more of the antiviral compounds identified herein for a sufficient amount of time under appropriate conditions to block binding of the virus to the host cell or host cell receptor.

The one or more antiviral compounds to be administered in accordance with the methods described herein inhibit the function of coronavirus protein, such as the interaction of the prodrug remdesivir with the RNA-directed RNA polymerase (RdRP); or the one or more antiviral compounds to be administered to infected humans or animals or combined with coronavirus organisms in accordance with the methods described herein is predicted to inhibit the function of coronavirus protease, or other crucial replication proteins, which in turn diminishes the ability for the coronavirus to replicate.

The one or more antiviral compounds are selected from the list of compounds provided below: Cinacalcet, Alverine, Alvimopan, Antazoline, Bepridil, Darifenacin, Desipramine, Diphenhydramine, Diphenidol, Flunarizine, Fosphenytoin, Furazolidone, Imipramine, Lifitegrast, Masitinib, Mifepristone, Nicardipine, Nilotinib, Olopatadine, Ospemifene, Oxprenolol, Perhexiline, Phenoxybenzamine, Phenylbutazone, Phenyltoloxamine, Pizotifen, Promazine, Stiripentol, Tamsulosin, Zolmitriptan, and salts thereof or combinations thereof.

### Antiviral Compounds

Suitable compounds for use in the methods described herein include the antiviral compounds shown below:

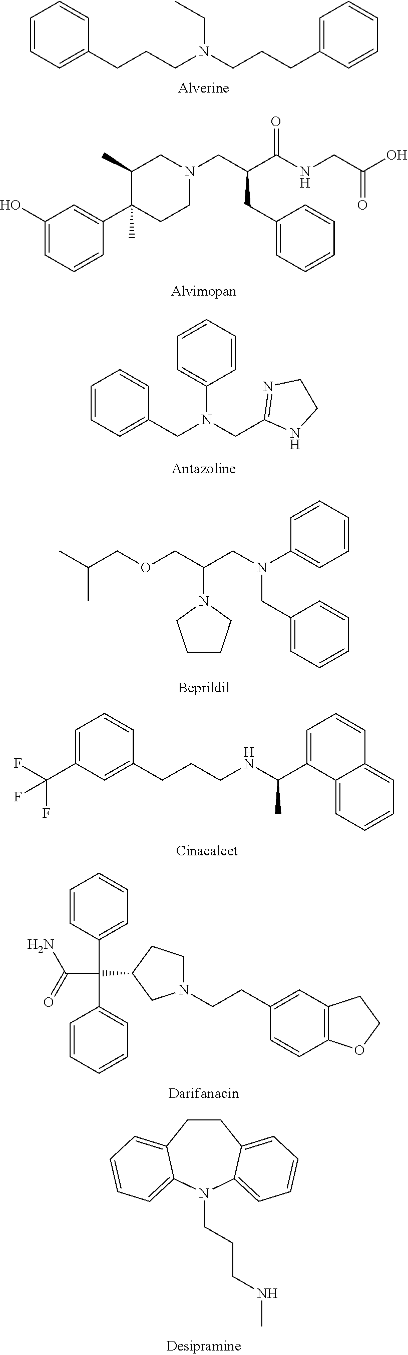

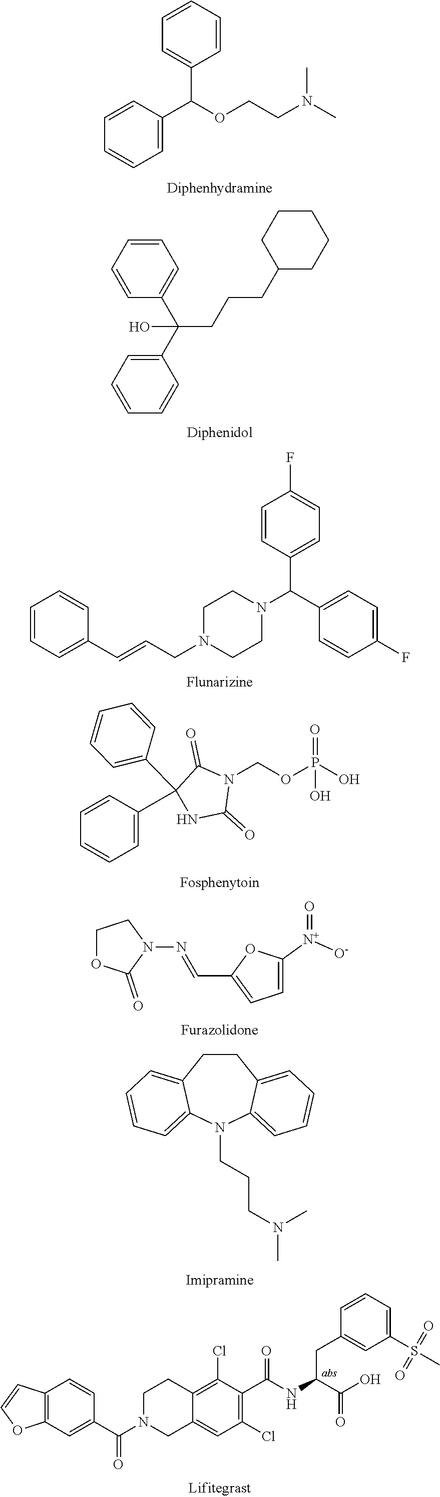

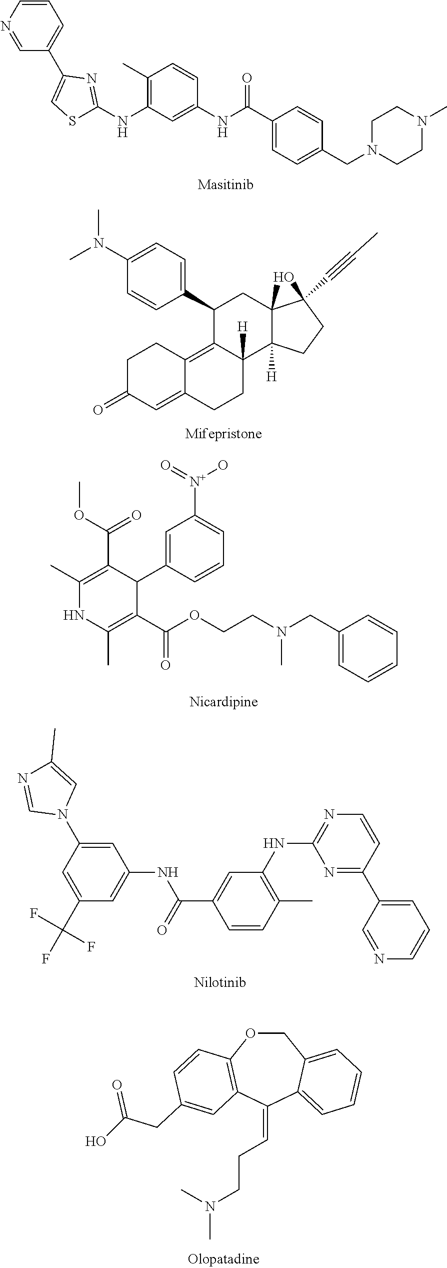

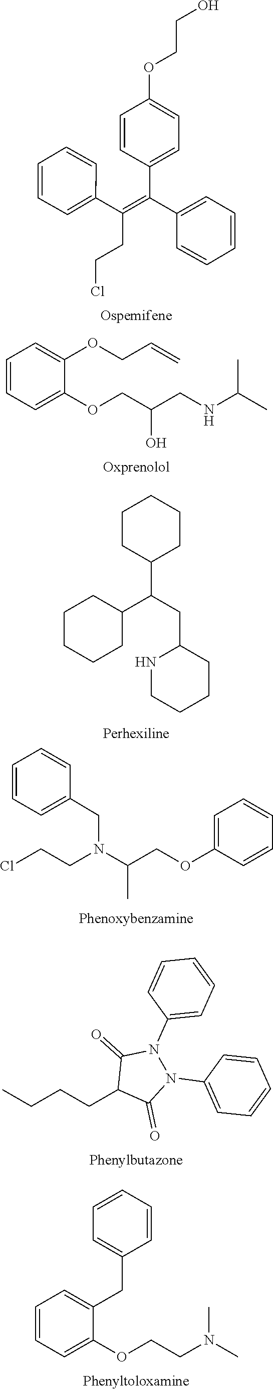

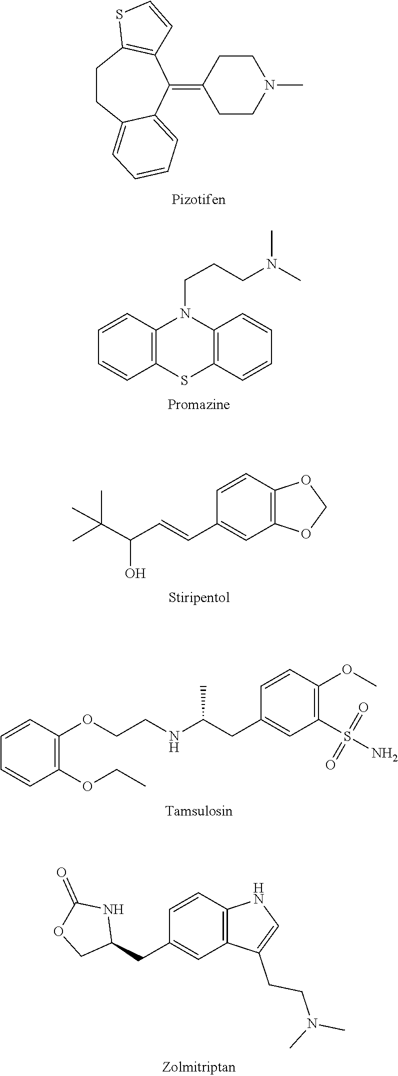 [SMILES: CN1CCC(=C2c3ccccc3CCc3sccc32)CC1 ; CN(C)CCCN1c2ccccc2Sc2ccccc21 ; CC(C)(C)C(O)/C=C/c1ccc2c(c1)OCO2 ; CCOc1ccccc1OCCN[C@H](C)Cc1ccc(OC)c(S(N)(=O)=O)c1 ; CN(C)CCc1c[nH]c2ccc(C[C@H]3COC(=O)N3)cc12]

The one or more antiviral compounds can also be selected from the list of exemplary compounds provided below, and in Tables 1-3, generated using pipelines within the Computational Analysis of Novel Drug Opportunities (CANDO) platform for shotgun drug discovery, repurposing, and design. Table 1 and Table 2 were generated using a de novo approach. Table 3 was based on (proteomic or fingerprint) similarity to remdesivir or favipiravir.

TABLE 1
3CLPro - SARS-CoV-2 3CL protease (main protease); PapainPro - SARS-CoV-2
papain-like protease; Approved - drug is approved for human use; CoV Inhibition -
experimental evidence exists in the literature that the compound inhibits the listed
coronaviruses.

| Name | Target(s) | Approved | CoV Inhibition |
|---|---|---|---|
| omacetaxine mepesuccinate | PapainPro; 3 CLPro | true | |
| mycophenolate_mofetil | 3CLPro; PapainPro | true | MERS; SARS-1 |
| pizotifen | PapainPro; 3 CLPro | true | |
| buprenorphine | 3CLPro; PapainPro | true | |
| stiripentol | PapainPro; 3 CLPro | true | |
| perhexiline | 3CLPro; PapainPro | true | SARS-2 |
| darifenacin | PapainPro; 3 CLPro | true | |
| alcaftadine | 3CLPro; PapainPro | true | |
| dihydro-alpha-ergocryptine | PapainPro; 3 CLPro | true | |
| gliquidone | PapainPro; 3 CLPro | true | |
| protokylol | PapainPro; 3 CLPro | true | |
| zeranol | 3CLPro; PapainPro | true | |
| guanoxan | PapainPro; 3 CLPro | true | |
| ulipristal | PapainPro; 3 CLPro | true | |
| mifepristone | PapainPro; 3 CLPro | true | |
| tam sul o sin | 3CLPro | true | |
| promazine | 3CLPro | true | MERS; SARS-1 |
| zolmitriptan | PapainPro; 3 CLPro | true | |
| ol op atadine | PapainPro; 3 CLPro | true | |
| dronabinol | 3CLPro; PapainPro | true | |
| dehydroemetine | 3 CLPro; PapainPro | false | |
| furazolidone | PapainPro; 3 CLPro | true | |
| bepridil (3CLPro score 0.98; found | 3CLPro | true | |
| to inhibit after prediction) | | | |
| perazine | 3CLPro | true | |
| imipramine | 3CLPro | true | |
| phenoxyb enzamine | 3CLPro | true | |
| alverine | 3CLPro | true | |
| dextropropoxyphene | 3CLPro | true | |
| alvimopan | 3CLPro | true | |
| methadone | 3CLPro | true | |
| nicardipine | 3CLPro | true | |
| antazoline | 3CLPro | true | |
| fosphenytoin | 3CLPro | true | |
| desipramine | 3CLPro | true | MERS; SARS-1 |
| phenylbutazone | 3CLPro | true | |
| diphenhydramine | 3CLPro | true | |
| diphenidol | 3CLPro | true | |
| fosamprenavir | 3CLPro | true | |
| amphetamine | 3CLPro | true | |
| hydralazine | 3CLPro | true | |
| thiabendazole | 3CLPro | true | |
| zonisamide | 3CLPro | true | |
| phenindione | 3CLPro | true | |
| dalfampridine | 3CLPro | true | |
| cyclobenzaprine | 3CLPro | true | |
| flunarizine | 3CLPro | true | |
| carisoprodol | 3CLPro | true | |
| paroxetine | PapainPro | true | |
| floctafenine | 3CLPro | true | |
| nitrazepam | 3CLPro | true | |
| levofloxacin | PapainPro | true | |
| ketoconazole | PapainPro | true | SARS-2 |
| nicotine | 3CLPro | true | |
| prucalopride | PapainPro | true | |
| methyltestosterone | 3CLPro; PapainPro | true | |
| racecadotril | 3CLPro | false | |
| delapril | 3CLPro | false | |
| quinaprilat | 3CLPro | false | |
| elesclomol | 3CLPro | false | |
| benactyzine | 3CLPro | false | |
| emopamil | 3CLPro | false | |
| batimastat | 3CLPro | false | |
| bms-906024 | 3CLPro | false | |
| trimethaphan | 3CLPro | true | |
| varespladib | 3CLPro | false | |
| febantel | 3CLPro | true | |
| dipipanone | 3CLPro | false | |
| bifemelane | 3CLPro | false | |
| benfluorex | 3CLPro | false | |
| fentanyl | 3CLPro | true | |
| remacemide | 3CLPro | false | |
| remdesivir | 3CLPro | false | MERS;SARS- |
| | | | 1;SARS-2 |
| darusentan | 3CLPro | false | |
| amprenavir | 3CLPro | true | |
| piritramide | 3CLPro | true | |
| ambrisentan | 3CLPro | true | |
| salvinorin a | 3CLPro | false | |
| eluxadoline | 3CLPro | true | |
| indecainide | 3CLPro | true | |
| oxaprozin | 3CLPro | true | |
| ragaglitazar | 3CLPro | false | |
| cinnamaldehyde | 3CLPro | true | |
| methsuximide | 3CLPro | true | |
| edaravone | 3CLPro | true | |
| pemoline | 3CLPro | true | |
| aminophenazone | 3CLPro | true | |
| antipyrine | 3CLPro | true | |
| disopyramide | 3CLPro | true | |
| cyclizine | 3CLPro | true | |
| pralidoxime | 3CLPro | true | |
| primidone | 3CLPro | true | |
| mephenytoin | 3CLPro | true | |
| glutethimide | 3CLPro | true | |
| phenobarbital | 3CLPro | true | |
| opipramol | 3CLPro | false | SARS-2 |
| toluene | 3CLPro | true | |
| methylphenobarbital | 3CLPro | true | |
| chloramphenicol succinate | 3CLPro | true | |
| pirfenidone | 3CLPro | true | |
| phenmetrazine | 3CLPro | true | |
| sarpogrelate | 3CLPro | false | |
| levamisole | 3CLPro | true | |
| phendimetrazine | 3CLPro | true | |
| coumarin | 3CLPro | false | |
| nisoxetine | 3CLPro | false | |
| tranylcypromine | 3CLPro | true | |
| 1-phenylcyclohexylamine | 3CLPro | false | |
| phencyclidine | 3CLPro | false | |
| aminorex | 3CLPro | false | |
| labetalol | 3CLPro | true | |
| atomoxetine | 3CLPro | true | |
| protriptyline | 3CLPro | true | |
| orphenadrine | 3CLPro | true | |
| menadione | 3CLPro | true | |
| tesmilifene | 3CLPro | false | |
| benzodiazepine | 3CLPro | true | |
| bucindolol | 3CLPro | false | |
| bretylium | 3CLPro | true | |
| phenethyl isothiocyanate | 3CLPro | false | |
| carvedilol | 3CLPro | true | |
| mephentermine | 3CLPro | true | |
| profenamine | 3CLPro | true | |
| tolazoline | 3CLPro | true | |
| pargyline | 3CLPro | true | |
| eplerenone | 3CLPro; PapainPro | true | |
| oxetacaine | 3CLPro | true | |
| zofenopril | 3CLPro | true | |
| carfentanil | 3CLPro | true | |
| benzphetamine | 3CLPro | true | |
| sacubitril | 3CLPro | true | |
| bumadizone | 3CLPro | true | |
| levacetylmethadol | 3CLPro | true | |
| selexipag | 3CLPro | true | |
| isopropamide | 3CLPro | true | |
| normethadone | 3CLPro | true | |
| mepenzolate | 3CLPro | true | |
| propantheline | 3CLPro | true | |
| medifoxamine | 3CLPro | true | |
| methantheline | 3CLPro | true | |
| benzydamine | 3CLPro | true | |
| darunavir | 3CLPro | true | |
| phenyltoloxamine | 3CLPro | true | |
| armodafinil | 3CLPro | true | |
| modafinil | 3CLPro | true | |
| diphenylpyraline | 3CLPro | true | |
| alpha-amyl cinnamaldehyde | 3CLPro | true | |
| cinnamyl alcohol | 3CLPro | true | |
| bendazac | 3CLPro | true | |
| pipradrol | 3CLPro | true | |
| phensuximide | 3CLPro | true | |
| fesoterodine | 3CLPro | true | |
| diphenylguanidine | 3CLPro | true | |
| ethotoin | 3CLPro | true | |
| dipyrithione | 3CLPro | true | |
| 1-benzylimidazole | 3CLPro | false | |
| safinamide | 3CLPro | true | |
| triamterene | 3CLPro | true | |
| tnp-470 | 3CLPro | false | |
| tadalafil | PapainPro | true | |
| rebamipide | 3CLPro | false | |
| cinoxacin | PapainPro | true | |
| tridihexethyl | 3CLPro | false | |
| doxazosin | PapainPro | true | MERS; SARS-1 |
| amoxapine | PapainPro | true | |
| valbenazine | 3CLPro | true | |
| phenelzine | 3CLPro | true | |
| oxolamine | 3CLPro | true | |
| alimemazine | 3CLPro | true | |
| butriptyline | 3CLPro | true | |
| oxybutynin | 3CLPro | true | |
| phenethylamine | 3CLPro | false | |
| quetiapine | 3CLPro | true | |
| phenothiazine | 3CLPro | true | |
| nomifensine | 3CLPro | true | |
| win 55212-2 | PapainPro | false | |
| enrasentan | PapainPro | false | |
| imidazole | 3CLPro | false | |
| nefopam | 3CLPro | true | |
| amobarbital | 3CLPro | true | |
| tubocurarine | PapainPro | true | |
| ginsenosides | 3CLPro | false | |
| isoaminile | 3CLPro | true | |
| dextroamphetamine | 3CLPro | true | |
| naftifine | 3CLPro | true | |
| thenalidine | 3CLPro | true | |
| propranolol | 3CLPro | true | MERS; SARS-1 |
| oxyphenonium | 3CLPro | true | |
| penbutolol | 3CLPro | true | |
| tacrine | 3CLPro | true | |
| idazoxan | 3CLPro; PapainPro | false | |
| resiniferatoxin | PapainPro; 3CLPro | false | |
| phentermine | 3CLPro | true | |
| pheniramine | 3CLPro | true | |
| nimesulide | 3CLPro | true | |
| thalidomide | 3CLPro | true | |
| clobazam | 3CLPro | true | |
| fotemustine | 3CLPro | false | |
| 3-bromo-7-nitroindazole | 3CLPro | false | |
| teniposide | PapainPro | true | |
| m-chlorophenylpiperazine | 3CLPro | false | |
| irinotecan | PapainPro; 3CLPro | true | |
| diprenorphine | 3CLPro; PapainPro | true | |
| methoxyphenamine | 3CLPro | true | |
| tetradecyl hydrogen sulfate (ester) | 3CLPro | true | |
| meprobamate | 3CLPro | true | |
| nortriptyline | 3CLPro | true | |
| metamfetamine | 3CLPro | true | |
| silodosin | 3CLPro | true | |
| phenytoin | 3CLPro | true | |
| antroquinonol | 3CLPro | false | |
| 7-nitroindazole | 3CLPro | false | |
| pivmecillinam | 3CLPro | true | |
| icotinib | PapainPro | false | |
| loxapine | PapainPro | true | |
| chlorcyclizine | 3CLPro | true | |
| dihydroergocornine | PapainPro | true | |
| dihydralazine | 3CLPro | true | |
| phosmet | 3CLPro | true | |
| mebutamate | 3CLPro | true | |
| diazepam | 3CLPro | true | |
| ketamine | 3CLPro | true | |
| belinostat | 3CLPro | true | |
| hydroxyzine | 3CLPro | true | |
| tesaglitazar | 3CLPro | false | |
| lapachone | 3CLPro | false | |
| atrasentan | PapainPro | false | |
| guanabenz | 3CLPro | true | |
| naproxcinod | 3CLPro | false | |
| naphazoline | 3CLPro | true | |
| tetrandrine | PapainPro | false | MERS; SARS- |
| | | | 1; SARS-2 |
| ethambutol | 3CLPro | true | |
| levmetamfetamine | 3CLPro | true | |
| atipamezole | 3CLPro | true | |
| trimipramine | 3CLPro | true | |
| florfenicol | 3CLPro | true | |
| rivaroxaban | PapainPro | true | |
| betahistine | 3CLPro | true | |
| esmolol | 3CLPro | true | |
| squalene | 3CLPro | true | |
| methapyrilene | 3CLPro | false | |
| piribedil | PapainPro | false | |
| talampanel | PapainPro | false | |
| nebivolol | PapainPro | true | |
| cytisine | 3CLPro | false | |
| dicyclomine | 3CLPro | true | |
| pacritinib | PapainPro | false | |
| 2-mercaptobenzothiazole | 3CLPro | true | |
| tetryzoline | 3CLPro | true | |
| levomenol | 3CLPro | true | |
| morpholinylmercaptobenzothiazole | 3CLPro | true | |
| ramelteon | PapainPro | true | |
| rasagiline | 3CLPro | true | |
| butylphthalide | 3CLPro | false | |
| st-101 | 3CLPro | false | |
| trandolapril | 3CLPro | true | |
| dalcetrapib | 3CLPro | false | |
| ticlopidine | 3CLPro | true | MERS; SARS-1 |
| terconazole | PapainPro | true | MERS; SARS-1 |
| cronidipine | PapainPro | false | |
| p-nitrobiphenyl | 3CLPro | true | |
| esketamine | 3CLPro | true | |
| ensulizole | 3CLPro | true | |
| oxyphencyclimine | 3CLPro | true | |
| ximelagatran | 3CLPro | true | |
| bromodiphenhydramine | 3CLPro | true | |
| elagolix | 3CLPro | true | |
| ifenprodil | 3CLPro | true | |
| tianeptine | 3CLPro | false | |
| cerulenin | 3CLPro | false | |
| lifitegrast | 3CLPro | true | |
| berberine | PapainPro | true | |
| sitaxentan | PapainPro | true | |
| pyrazole | 3CLPro | false | |
| epanolol | 3CLPro | false | |
| midomafetamine | PapainPro | false | |
| bicuculline | PapainPro | false | |
| icosapent ethyl | 3CLPro | true | |
| picropodophyllin | PapainPro | false | |
| tocotrienol | 3CLPro | false | |
| 4-(isopropylamino)diphenylamine | 3CLPro | true | |
| apronalide | 3CLPro | true | |
| piperine | PapainPro | false | |
| isoxsuprine | 3CLPro | true | |
| triapine | 3CLPro | false | |
| perindopril | 3CLPro | true | |
| podofilox | PapainPro | true | |
| nalfurafine | PapainPro | false | |
| doxylamine | 3CLPro | true | |
| flavone | 3CLPro | true | |
| hexocyclium | 3CLPro | true | |

TABLE 2

| | | | CoV |
|---|---|---|---|
| | | | Inhibi- |
| Name | Methods | Approved | tion |
| perazine | HP; FP; GH | true | |
| imipramine | HP; FP; GH | true | |
| promazine | HP; FP; GH | true | SARS-1; |
| | | | MERS |
| desipramine | HP | true | SARS-1; |
| | | | MERS |
| tamsulosin | HP; GH | true | |
| oxprenolol | HP; FP | true | |
| phenyltoloxamine | HP; FP; GH | true | |
| masitinib | HP; FP; GH | true | |
| ospemifene | HP; FP; GH | true | |
| nilotinib | HP | true | |
| astemizole | GH | true | |
| noscapine | FP; GH | true | |
| cutamesine | HP; FP | false | |
| trimethobenzamide | HP; FP; GH | true | |
| ivabradine | FP; GH | true | |
| pindolol | HP; FP | true | |
| thiazinam | HP; FP | false | |
| dimetacrine | HP; FP | false | |
| pipotiazine | HP; FP; GH | true | |
| zuclopenthixol | HP; FP; GH | true | |
| gamithromycin | GH | true | |
| prochlorperazine | HP; FP; GH | true | |
| chlorpromazine | HP; FP; GH | true | SARS-2; |
| | | | SARS-1; |
| | | | MERS |
| trifluoperazine | HP; FP; GH | true | |
| triflupromazine | HP; FP; GH | true | SARS-1; |
| | | | MERS |
| acepromazine | HP; FP; GH | true | |
| propiopromazine | HP; FP; GH | true | |
| clomipramine | HP; FP; GH | true | SARS-2; |
| | | | SARS-1; |
| | | | MERS |
| perphenazine | HP; FP; GH | true | |
| thiethylperazine | HP; FP; GH | true | SARS-2; |
| | | | SARS-1; |
| | | | MERS |
| dimetotiazine | HP; FP; GH | true | |
| methotrimeprazine | HP; FP; GH | true | |
| amodiaquine | HP; FP; GH | true | SARS-2; |
| | | | SARS-1; |
| | | | MERS |
| flupentixol | HP; FP; GH | true | |
| hydroxychloroquine | HP; FP; GH | true | SARS-2; |
| | | | SARS-1; |
| | | | MERS |
| chloroquine | HP; FP; GH | true | SARS-2; |
| | | | SARS-1; |
| | | | MERS |
| clomifene | HP; FP; GH | true | |
| chlorproethazine | HP; FP | false | |
| thioproperazine | HP; FP | false | |
| chlorprothixene | FP; GH | true | |
| lofepramine | HP; FP | false | |
| carphenazine | HP; FP | false | |
| imipramine_oxide | HP; FP | false | |
| clocapramine | HP; FP | false | |
| butaperazine | HP; FP | true | |
| thiopropazate | HP; FP | false | |
| cyamemazine | HP; FP | false | |
| quinacrine | HP; FP | false | |
| cinchocaine | FP; GH | true | |
| quinacrine_mustard | HP; FP | false | |
| pimozide | FP; GH | true | |
| afimoxifene | HP; FP | false | |
| fispemifene | HP; FP | false | |
| chlorotoxin_i-131 | HP; FP | false | |
| 4-[(4-methyl-1-piperazinyl) | HP; FP | false | |
| methyl]-n-[3-[[4-(3-pyridinyl)- | | | |
| 2-pyrimidinyl]amino]phenyl]- | | | |
| benzamide | | | |
| neratinib | HP; GH | true | |
| fluphenazine | FP; GH | true | SARS-2; |
| | | | SARS-1; |
| | | | MERS |
| tetrahydropalmatine | HP; FP | false | |
| enclomiphene | HP; FP | false | |
| prenalterol | HP; FP | false | |
| tubocurarine | FP; GH | true | |
| practolol | HP; FP | true | |
| ibutilide | HP; FP | true | |
| metoprolol | HP; FP | true | |
| clopenthixol | HP; FP | false | |
| quinupramine | FP; GH | true | |
| 4-{2-[4-(2-aminoethyl)piperazin- | HP; FP | false | |
| 1-yl]pyridin-4-yl}-n-(3-chloro-4- | | | |
| methylphenyl)pyrimidin-2-amine | | | |
| dimenoxadol | HP; FP | false | |
| dubermatinib | HP; FP | false | |
| bisoprolol | HP; FP | true | |
| befunolol | HP; FP | false | |
| rupatadine | HP; GH | true | |
| trifluperidol | HP; FP | false | |
| acetophenazine | FP | true | |
| rilpivirine | GH | true | |
| entrectinib | GH | true | |
| flurazepam | GH | true | |
| macimorelin | GH | true | |
| prothipendyl | FP | false | |
| setiptiline | HP | false | |
| capmatinib | GH | true | |
| toremifene | GH | true | |
| irinotecan | GH | true | |
| spiramycin | GH | true | |
| zotepine | GH | true | |
| azelastine | GH | true | |
| flunarizine | GH | true | |
| oleandomycin | GH | true | |
| erythromycin | GH | true | |
| sitamaquine | FP | false | |
| albaconazole | FP | false | |
| alimemazine | FP | true | |
| perphenazine_enanthate | FP | false | |
| ritonavir | GH | true | |
| cobicistat | GH | true | |
| ribociclib | GH | true | |
| isavuconazole | GH | true | |
| gilteritinib | GH | true | SARS-2 |
| thonzonium | GH | true | |
| zolpidem | GH | true | |
| brilliant_green_cation | GH | true | |
| acetyldigitoxin | GH | true | |
| deracoxib | GH | true | |
| antrafenine | GH | true | |
| butenafine | GH | true | |
| palbociclib | GH | true | |
| betrixaban | GH | true | |
| meclizine | GH | true | |
| piperaquine | FP | false | |
| ibrutinib | GH | true | |
| etoricoxib | GH | true | |
| clarithromycin | GH | true | |
| vemurafenib | GH | true | |
| etymemazine | GH | true | |
| axitinib | GH | true | |
| letrozole | GH | true | |
| indinavir | GH | true | |
| arzoxifene | GH | true | |
| 3-[3-chloro-5-(5-{[(1s)-1- | HP | false | |
| phenylethyl]amino}isoxazolo | | | |
| [5_4-c]pyridin-3-yl)phenyl] | | | |
| propan-1-ol | | | |
| azithromycin | GH | true | SARS-2 |
| ouabain | GH | true | SARS-2 |
| simeprevir | GH | true | |
| saquinavir | GH | true | SARS-2 |
| gentamicin | GH | true | |
| netilmicin | GH | true | |
| tolnaftate | GH | true | |
| citalopram | GH | true | |
| lasofoxifene | GH | true | |
| midazolam | GH | true | |
| cinacalcet | GH | true | |
| dirlotapide | GH | true | |
| pyrvinium | GH | true | |
| almitrine | GH | true | SARS-2 |
| caspofungin | GH | true | |
| estazolam | GH | true | |
| aminopromazine | GH | true | |
| gentian_violet_cation | GH | true | |
| benzquinamide | FP | false | |
| risperidone | GH | true | |
| drometrizole_trisiloxane | GH | true | |
| (1-benzyl-5-methoxy-2-methyl- | HP | false | |
| 1h-indol-3-yl)acetic_acid | | | |
| trimipramine | FP | true | |
| itopride | FP | false | |
| valbenazine | FP | true | |
| mosapramine | FP | false | |
| acotiamide | HP | false | |
| metopimazine | FP | false | |
| dibenzepin | HP | true | |
| camylofin | FP | false | |
| (2r)-1-[4-({4-[(2_5- | FP | false | |
| dichlorophenyl)amino]-2- | | | |
| pyrimidinyl}amino)phenoxy]-3- | | | |
| (dimethylamino)-2-propanol | | | |
| opipramol | FP | false | |
| (2s)-1-[4-({4-[(2_5- | FP | false | |
| dichlorophenyl)amino]-2- | | | |
| pyrimidinyl}amino)phenoxy]-3- | | | |
| (dimethylamino)-2-propanol | | | |
| dixyrazine | FP | false | |
| promethazine | FP | true | SARS-1; |
| | | | MERS |
| methdilazine | FP | true | |
| etoperidone | HP | false | |
| alverine | FP | true | |
| docosanol | GH | true | |
| colistimethate | GH | true | |
| tamoxifen | GH | true | SARS-2 |
| distearyldimonium | GH | true | |
| cetalkonium | GH | true | |
| bicalutamide | GH | true | |
| terconazole | GH | true | |
| diethylamino_hydroxy- | GH | true | |
| benzoyl_hexyl_benzoate | | | |
| lopinavir | GH | true | SARS-2 |
| repaglinide | GH | true | |
| plecanatide | GH | true | |
| diphenoxylate | GH | true | |
| atazanavir | GH | true | SARS-2 |
| technetium_tc-99m_tetrofosmin | GH | true | |
| temazepam | GH | true | |
| propiomazine | GH | true | |
| ebastine | GH | true | SARS-2 |
| hydrocortisone_probutate | GH | true | |
| itraconazole | GH | true | |
| venetoclax | GH | true | |
| vismodegib | GH | true | |
| fesoterodine | GH | true | |
| (2s)-1-(9h-carbazol-4-yloxy)-3- | FP | false | |
| (isopropylamino)propan-2-ol | | | |
| nonoxynol-9 | GH | true | |
| pirfenidone | GH | true | |
| dactinomycin | GH | true | |
| dabigatran_etexilate | GH | true | |
| avatrombopag | GH | true | SARS-2 |
| dehydroemetine | FP | false | |
| dioxaphetyl_butyrate | FP | false | |
| ubiquinone-2 | FP | false | |
| digitoxin | GH | true | SARS-2 |
| tetrofosmin | GH | true | |
| domiphen | GH | true | |
| benzododecinium | GH | true | |
| methylene_blue | GH | true | SARS-2 |
| erdafitinib | GH | true | |
| idelalisib | GH | true | |
| asenapine | GH | true | |
| berberine | GH | true | |
| lidocaine | GH | true | |
| tolterodine | GH | true | |
| raloxifene | GH | true | |
| fluspirilene | GH | true | SARS-2 |
| doxepin | GH | true | |
| fexofenadine | GH | true | |
| tetrabenazine | FP | true | |
| dichlorophen | FP | true | |
| tg-100801 | HP | false | |
| zaleplon | GH | true | |
| cetyl_alcohol | GH | true | |
| colistin | GH | true | |
| isopropamide | GH | true | |
| linagliptin | GH | true | |
| metixene | GH | true | |
| haloperidol | FP | true | |
| piroxicam | GH | true | |
| ponatinib | GH | true | |
| lorpiprazole | GH | true | |
| difenoxin | GH | true | |
| tritoqualine | FP | true | |
| mepindolol | FP | false | |
| didecyldimethylammonium | GH | true | |
| sofosbuvir | GH | true | |
| eltrombopag | GH | true | SARS-2 |
| apremilast | GH | true | |
| darolutamide | GH | true | |
| pimavanserin | GH | true | |
| demecarium | GH | true | |
| daptomycin | GH | true | |
| bacitracin | GH | true | |
| buclizine | GH | true | |
| amiodarone | GH | true | |
| mebeverine | GH | true | |
| escitalopram | GH | true | |
| suvorexant | GH | true | |
| bicuculline | FP | false | |
| distigmine | HP | false | |
| mgb-bp-3 | HP | false | |
| febantel | GH | true | |
| ketazolam | GH | true | |
| revefenacin | GH | true | |
| bepridil | GH | true | |
| thioridazine | GH | true | SARS-2 |
| boscalid | GH | true | |
| hydroxyzine | GH | true | |
| phenoxybenzamine | GH | true | |
| naftifine | GH | true | |
| vindesine | GH | true | |
| thiothixene | GH | true | |
| lidoflazine | FP | false | |
| deslanoside | GH | true | |
| miltefosine | GH | true | |
| oxybenzone | GH | true | |
| telmisartan | GH | true | |
| dosulepin | GH | true | |
| darifenacin | GH | true | |
| indenolol | FP | false | |
| tenoxicam | GH | true | |
| benzonatate | GH | true | |
| candesartan_cilexetil | GH | true | |
| ethylhexyl_methoxycrylene | GH | true | |
| osimertinib | GH | true | SARS-2 |
| amsacrine | GH | true | |
| mianserin | GH | true | |
| carfilzomib | GH | true | |
| primaquine | FP | true | |
| vanoxerine | FP | false | |
| profenamine | FP | true | |
| paliroden | HP | false | |
| cetrimonium | GH | true | |
| fusidic_acid | GH | true | |
| acenocoumarol | GH | true | |
| carfentanil | GH | true | |
| parecoxib | GH | true | |
| prednisolone_tebutate | GH | true | |
| cinnarizine | GH | true | |
| fentanyl | GH | true | |
| penfluridol | FP | false | |
| hki-357 | HP | false | |
| 6-[n-(1-isopropyl-3_4-dihydro- | HP | false | |
| 7-isoquinolinyl)carbamyl]-2- | | | |
| naphthalenecarboxamidine | | | |
| tiamulin | GH | true | |
| torasemide | GH | true | |
| naldemedine | GH | true | |
| ambenonium | GH | true | |
| meclofenoxate | FP | false | |
| cadralazine | HP | false | |
| dacomitinib | GH | true | |
| sodium_1_2-dipalmitoyl-sn- | GH | true | |
| glycero-3-phospho-(1′-rac- | | | |
| glycerol) | | | |
| pazopanib | GH | true | |
| pemirolast | GH | true | |
| lemborexant | GH | true | |
| nuc-1031 | FP | false | |
| tesmilifene | FP | false | |
| moperone | FP | false | |
| flumatinib | HP | false | |
| polymyxin_b | GH | true | |
| larotrectinib | GH | true | |
| lomitapide | GH | true | |
| carmegliptin | FP | false | |
| 1-(2-phenylethyl)-4-phenyl-4- | HP | false | |
| acetoxypiperidine | | | |
| 1-palmitoyl-2-oleoyl-sn- | GH | true | |
| glycero-3-(phospho-rac-(1- | | | |
| glycerol)) | | | |
| bromperidol | FP | true | |
| alprenolol | HP | false | SARS-1; |
| | | | MERS |
| halofantrine | FP | true | |
| isothipendyl | FP | false | |
| 5-[1-(3_4-dimethoxy-benzoyl)- | HP | false | |
| 1_2_3_4-tetrahydro-quinolin- | | | |
| 6-yl]-6-methyl-3_6-dihydro- | | | |
| [1_3_4]thiadiazin-2-one | | | |
| chlorproguanil | HP | false | |
| azeliragon | FP | false | |
| taladegib | HP | false | |
| (1r)-n_6-dihydroxy-7-methoxy- | FP | false | |
| 2-[(4-methoxyphenyl)sulfonyl]- | | | |
| 1_2_3_4-tetrahydroisoquinoline- | | | |
| 1-carboxamide | | | |
| 2-(2_4-dichlorophenoxy)-5- | HP | false | |
| (pyridin-2-ylmethyl)phenol | | | |
| n-(2-hydroxy-1_1-dimethylethyl)- | HP | false | |
| 1-methyl-3-(1h-pyrrolo[2_3-b] | | | |
| pyridin-2-yl)-1h-indole-5- | | | |
| carboxamide | | | |
| tretoquinol | FP | false | |
| 4-(4-chloro-phenyl)-1-{3-[2-(4- | FP | false | |
| fluoro-phenyl)-[1_3]dithiolan-2- | | | |
| yl]-propyl}-piperidin-4-ol | | | |
| mk-7145 | FP | false | |
| 3-(3_4-dimethoxyphenyl) | FP | false | |
| propionic_acid | | | |
| zk-806450 | HP | false | |
| florbenazine_f-18 | FP | false | |
| diphenhydramine | FP | true | |
| periciazine | FP | true | |
| atenolol | FP | true | |
| bephenium | HP | false | |
| nabumetone | HP | true | |
| (4r)-4-(3-butoxy-4- | FP | false | |
| methoxybenzyl)imidazolidin- | | | |
| 2-one | | | |
| selexipag | HP | true | |
| (r)-atenolol | FP | false | |
| deutetrabenazine | FP | true | |
| phenoxyethanol | FP | true | |
| aceprometazine | FP | false | |
| metipranolol | FP | true | |
| gsk-376501 | HP | false | |
| 4-[4-(1-amino-1-methylethyl) | HP | false | |
| phenyl]-5-chloro-n-[4-(2- | | | |
| morpholin-4-ylethyl)phenyl] | | | |
| pyrimidin-2-amine | | | |
| almorexant | FP | false | |
| declopramide | FP | false | |
| normethadone | FP | true | |
| pimethixene | FP | true | |
| esatenolol | FP | false | |
| 2-(3-bromophenyl)-6-[(2- | HP | false | |
| hydroxyethyl)amino]-1h- | | | |
| benzo[de]isoquinoline- | | | |
| 1_3(2h)-dione | | | |
| 4-{4-[(5-hydroxy-2- | FP | false | |
| methylphenyl)amino]quinolin- | | | |
| 7-yl}-1_3-thiazole-2- | | | |
| carbaldehyde | | | |
| pbt-1033 | FP | false | |
| 10_11-dimethoxy-4- | FP | false | |
| methyldibenzo[c_f]-2_7- | | | |
| naphthyridine-3_6-diamine | | | |
| tmc-647055 | HP | false | |
| ropinirole | HP | true | |
| 2-(3- | FP | false | |
| benzoylphenoxy)ethyl(hydroxy) | | | |
| formamide | | | |
| meptazinol | HP | false | |
| chlorphenamine | FP | true | |
| fludiazepam | HP | false | |
| adinazolam | HP | false | |
| naftidrofuryl | HP | false | |
| dexchlorpheniramine | FP | false | |
| lumefantrine | FP | true | |
| clebopride | FP | false | |
| esmolol | FP | true | |
| acebutolol | FP | true | |
| (2z)-2-cyano-n-(3′- | HP | false | |
| ethoxybiphenyl-4-yl)-3- | | | |
| hydroxybut-2-enamide | | | |
| doxylamine | FP | true | |
| pilsicainide | FP | false | |
| almotriptan | HP | true | |
| ispinesib | FP | false | |
| meclinertant | FP | false | |
| cloranolol | HP | false | |
| celiprolol | HP | true | |
| chlorphenoxamine | FP | false | |
| amitriptyline | FP | true | |
| n-cyclopropyl-6-[(6_7- | FP | false | |
| dimethoxyquinolin-4- | | | |
| yl)oxy]naphthalene-1- | | | |
| carboxamide | | | |
| vonoprazan | HP | false | |
| florbetaben_(18f) | HP | true | |
| sgx-523 | HP | false | |
| penbutolol | FP | true | |
| 4-[(3r)-3-{[2-(4-fluorophenyl)-2- | HP | false | |
| oxoethyl]amino}butyl]benzamide | | | |
| moxifloxacin | HP | true | |
| n-(5-isopropyl-thiazol-2-yl)-2- | HP | false | |
| pyridin-3-yl-acetamide | | | |
| evodenoson | HP | false | |
| vernakalant | FP | true | |
| guaifenesin | FP | true | |
| tinostamustine | HP | false | |
| bromodiphenhydramine | FP | true | |
| tariquidar | FP | false | |
| fipexide | FP | false | |
| mephenesin | FP | true | |
| clonazolam | HP | false | |
| 4-[(5-{[4-(3-chlorophenyl)- | HP | false | |
| 3-oxopiperazin-1- | | | |
| yl]methyl}-1h-imidazol-1- | | | |
| yl)methyl]benzonitrile | | | |
| donepezil | FP | true | |
| propamidine | FP | false | |
| fluanisone | FP | false | |
| domperidone | HP | true | |
| funapide | HP | false | |
| orphenadrine | FP | true | |
| etoxeridine | FP | false | |
| psi-352938 | HP | false | |
| (3as)-3a-hydroxy-7-methyl-1- | HP | false | |
| phenyl-1_2_3_3a-tetrahydro-4h- | | | |
| pyrrolo[2_3-b]quinolin-4-one | | | |
| duvelisib | GH | true | |
| steviolbioside | GH | true | |
| digoxin | GH | true | SARS-2 |
| narasin | GH | true | |
| tolcapone | GH | true | |
| lormetazepam | GH | true | |
| ruxolitinib | GH | true | |
| benorilate | GH | true | |
| didanosine | GH | true | |
| tenofovir_alafenamide | GH | true | |
| adenosine_phosphate | GH | true | |
| haloprogin | GH | true | |
| drotaverine | GH | true | SARS-2 |
| regorafenib | GH | true | SARS-2 |
| sorafenib | GH | true | SARS-2 |
| ethyl_salicylate | GH | true | |
| mercaptopurine* | GH | true | |
| doramectin | GH | true | |
| amitraz | GH | true | |
| desoxycorticosterone_pivalate | GH | true | |
| metocurine_iodide | GH | true | |
| cyclosporine | GH | true | SARS-2 |
| dofetilide | GH | true | |
| amcinonide | GH | true | |
| micafungin | GH | true | |
| flavone | GH | true | |
| tepoxalin | GH | true | |
| verapamil | GH | true | |
| dinoprostone | GH | true | |
| sertindole | GH | true | |
| papaverine | GH | true | SARS-2 |
| gestonorone_caproate | GH | true | |
| cevimeline | GH | true | |
| mesoridazine | GH | true | |
| monensin | GH | true | |
| triclocarban | GH | true | |
| lorazepam | GH | true | |
| eszopiclone | GH | true | |
| bezafibrate | GH | true | |
| entecavir | GH | true | |
| citicoline | GH | true | |
| chloroxine | GH | true | |
| irbesartan | GH | true | |
| oseltamivir | GH | true | |
| methyl_salicylate | GH | true | |
| flucytosine | GH | true | |
| spinosad | GH | true | |
| artemether | GH | true | |
| isocarboxazid | GH | true | |
| benzhydrocodone | GH | true | |
| beclomethasone_dipropionate | GH | true | |
| metocurine | GH | true | |
| olmesartan | GH | true | |
| desoxycorticosterone_acetate | GH | true | |
| vancomycin | GH | true | |
| phenothiazine | GH | true | |
| unoprostone | GH | true | |
| testosterone_enanthate | GH | true | |
| lynestrenol | GH | true | |
| loperamide | GH | true | SARS-2 |
| isoxaflutole | GH | true | |
| lumacaftor | GH | true | |
| spectinomycin | GH | true | |
| pipecuronium | GH | true | |
| triclosan | GH | true | |
| reserpine | GH | true | |
| tadalafil | GH | true | |
| zopiclone | GH | true | |
| lamivudine | GH | true | |
| azacitidine | GH | true | |
| clioquinol | GH | true | |
| peramivir | GH | true | |
| choline_magnesium_trisalicylate | GH | true | |
| adenine | GH | true | |
| mazindol | GH | true | |
| selamectin | GH | true | |
| naftazone | GH | true | |
| trazodone | GH | true | |
| megestrol_acetate | GH | true | |
| retapamulin | GH | true | |
| oritavancin | GH | true | |
| phenytoin | GH | true | |
| epoprostenol | GH | true | |
| letermovir | GH | true | |
| testosterone_undecanoate | GH | true | |
| rilmenidine | GH | true | |
| terfenadine | GH | true | |
| solifenacin | GH | true | |
| lansoprazole | GH | true | |
| oxandrolone | GH | true | |
| dasatinib | GH | true | |
| benzatropine | GH | true | |
| dexlansoprazole | GH | true | |

CANDO predicted candidates based on:
HP—drug-proteome (human) interaction signatures;
FP—drug-molecular fingerprint signatures;
GH—drug-proteome (human) interaction signatures;
Approved—drug is approved for human use;
CoV Inhibition—experimental evidence exists in the literature that the compound inhibits the listed coronaviruses.

TABLE 3

| | | | CoV |
|---|---|---|---|
| Name | Similar NTPs | Approved | Inhibition |
| zidovudine_monophosphate | remdesivir; favipiravir | false | |
| 2′_3′-dideoxy-3′-fluoro-urididine-5′- | remdesivir; favipiravir | false | |
| diphosphate | | | |
| 3′-thio-thymidine-5′-phosphate | remdesivir; favipiravir | false | |
| phosphoric_acid_mono-[3-fluoro-5- | remdesivir; favipiravir | false | |
| (5-methyl-2_4-dioxo-3_4-dihydro- | | | |
| 2h-pyrimidin-1-yl)-tetrahyro-furan-2- | | | |
| ylmethyl]_ester | | | |
| remdesivir_nucleoside_diphosphate | remdesivir; favipiravir | false | |
| 2′-o-methyl-3′-methyl-3′-deoxy- | remdesivir; favipiravir | false | |
| arabinofuranosyl-thymine-5′-phosphate | | | |
| deoxyuridine-5′-triphosphate | remdesivir; favipiravir | false | |
| guanosine_5′-diphosphate_2′:3′- | remdesivir; favipiravir | false | |
| cyclic_monophosphate | | | |
| 3′-o-acetylthymidine-5′-diphosphate | remdesivir; favipiravir | false | |
| 3′-deoxy-3′-aminothymidine_monophosphate | remdesivir; favipiravir | false | |
| 8-oxo-dgmp | remdesivir; favipiravir | false | |
| 2′-deoxyuridine_5′-alpha_beta-imido- | remdesivir; favipiravir | false | |
| triphosphate | | | |
| cordycepin_triphosphate | remdesivir; favipiravir | false | |
| phenyl-uridine-5′-diphosphate | remdesivir; favipiravir | false | |
| lamivudine-triphosphate | remdesivir; favipiravir | false | |
| 2′-deoxyguanosine-5′-diphosphate | remdesivir; favipiravir | false | |
| gemcitabine_triphosphate | remdesivir; favipiravir | false | |
| 2′-deoxycytidine_5′-triphosphate | remdesivir; favipiravir | false | |
| histidyl-adenosine_monophosphate | remdesivir; favipiravir | false | |
| gs-606965 | remdesivir; favipiravir | false | |
| {[2-amino-4-oxo-6_7-di(sulfanyl- | remdesivir; favipiravir | false | |
| CE∫s)-3_5_5a_8_9a_10-hexahydro- | | | |
| 4h-pyrano[3_2-g]pteridin-8- | | | |
| yl]methyl_dihydrogenato(2- | | | |
| )_phosphate}(dioxo)sulfanylmolybdenum | | | |
| 3′-azido-3′-deoxythymidine-5′- | remdesivir; favipiravir | false | |
| diphosphate | | | |
| 2′_3′-dehydro-2′_3′-deoxy- | remdesivir; favipiravir | false | |
| thymidine_5′-triphosphate | | | |
| 2′_3′-o-{4-[hydroxy(oxido)-CE^{a}5- | remdesivir; favipiravir | false | |
| azanylidene[-2_6-dinitro-2_5- | | | |
| cyclohexadiene-1_1-diyl}adenosine_5′- | | | |
| (tetrahydrogen_triphosphate) | | | |
| thymidine-5′-_diphosphate | remdesivir; favipiravir | false | |
| thymidine-5′-triphosphate | remdesivir; favipiravir | false | |
| puromycin_aminonucleoside-5′- | remdesivir; favipiravir | false | |
| monophosphate | | | |
| 2′_3′-dehydro-2′_3′-deoxy- | remdesivir; favipiravir | false | |
| thymidine_5′-diphosphate | | | |
| gs-461203 | remdesivir; favipiravir | false | |
| phosporic_acid_mono-[3_4- | remdesivir; favipiravir | false | |
| dihydroxy-5-(5-methoxy- | | | |
| benzoimidazol-1-yl)-tetrahydro- | | | |
| furan-2-ylmethyl]_ester | | | |
| 2′-deoxyguanosine-5′-monophosphate | remdesivir; favipiravir | false | |
| thymidine-3′_5′-diphosphate | remdesivir; favipiravir | false | |
| 2-chloro-2′-deoxyadenosine-5′-triphosphate | remdesivir; favipiravir | false | |
| clofarabind-5′-monophosphate | remdesivir; favipiravir | false | |
| hypoxanthine | favipiravir | false | |
| 2′-deoxyuridine_5′-alpha_beta-imido- | remdesivir; favipiravir | false | |
| diphosphate | | | |
| 3′-phosphate-adenosine-5′- | remdesivir; favipiravir | false | |
| phosphate_sulfate | | | |
| n6-benzyl_adenosine-5′-diphosphate | remdesivir | false | |
| 2′-deoxyguanosine-5′-triphosphate | remdesivir; favipiravir | false | |
| 1-(2-deoxy-5-o-phosphono-beta-d- | remdesivir; favipiravir | false | |
| erythro-pentofuranosyl)-5-nitro-1h-indole | | | |
| gemcitabine_monophosphate | remdesivir; favipiravir | false | |
| brivudine_monophosphate | remdesivir; favipiravir | false | |
| 3′-deoxy_3′-amino_adenosine-5′-diphosphate | remdesivir; favipiravir | false | |
| [(2r-3r-4s-5s)-3-4-dihydroxy-5-(4- | remdesivir; favipiravir | false | |
| oxo-4-5-dihydro-1h-pyrrolo[3_2- | | | |
| d]pyrimidin-7-yl)-2-pyrrolidinyl] | | | |
| methyl_dihydrogen_phosphate | | | |
| 6-aminohexyl-uridine-c1_5′-diphosphate | remdesivir; favipiravir | false | |
| prednisolone_phosphate | remdesivir; favipiravir | true | |
| 2-methylthio-n6-isopentenyl- | remdesivir; favipiravir | false | |
| adenosine-5′-monophosphate | | | |
| tryptophanyl-5′amp | remdesivir | false | |
| alpha-ribazole-5′-phosphate_derivative | remdesivir; favipiravir | false | |
| adenosine-3′-5′-diphosphate | remdesivir; favipiravir | false | |
| adenosine-2′-5′-diphosphate | remdesivir; favipiravir | false | |
| 2′_3′-dideoxyadenosine-5′-triphosphate | remdesivir; favipiravir | false | |
| 2′-o-[1-ethyl-1h- | remdesivir; favipiravir | false | |
| imidazol)]_thymidine-5′-monophosphate | | | |
| thiodeoxyguanosine_diphosphate | remdesivir; favipiravir | false | |
| thymidine_monophosphate | remdesivir; favipiravir | false | |
| 2′-deoxyuridine_3′-monophosphate | remdesivir; favipiravir | false | |
| deoxyuridine_monophosphate | remdesivir; favipiravir | false | |
| rovafovir_etalafenamide | remdesivir | false | |
| pyrazinamide | favipiravir | true | |
| remdesivir_nucleoside_monophosphate | remdesivir | false | |
| ethyl_2-amino-4-hydroxypyrimidine-5- | favipiravir | false | |
| carboxylate | | | |
| 5-hydroxypyrazinamide | favipiravir | false | |
| bms-986094 | remdesivir | false | |
| uprifosbuvir | remdesivir | false | |
| nuc-1031 | remdesivir | false | |
| pyrazinoic_acid | favipiravir | false | |
| 1-(2-deoxy-5-o-phosphono-beta-d-erythro- | remdesivir | false | |
| pentofuranosyl)-4-methyl-1h-indole | | | |
| tenofovir_alafenamide | remdesivir | true | |
| 5-bromonicotinamide | favipiravir | false | |
| sofosbuvir | remdesivir | true | |
| gs-6620 | remdesivir | false | |
| favipiravir_nucleoside_triphosphate | remdesivir | false | |
| thymectacin | remdesivir | false | |
| trans-urocanic_acid | favipiravir | false | |
| amiloride | favipiravir | true | |
| regrelor | remdesivir | false | |
| n-naphthalen-1-ylmethyl-2′-[3_5- | remdesivir | false | |
| dimethoxybenzamido]-2′-deoxy-adenosine | | | |
| 3′-oxo-adenosine | remdesivir | false | |
| 4-chloro-5-sulfamoylanthranilic_acid | favipiravir | false | |
| 1_2_4-triazole-carboxamidine | favipiravir | false | |
| adafosbuvir | remdesivir | false | |
| dacabazine | favipiravir | true | |
| dabigatran_2-o-acylglucuronide_metabolite | remdesivir | false | |
| dabigatran_3-o-acylglucuronide_metabolite | remdesivir | false | |
| flucytosine | favipiravir | true | |
| lobucavir | remdesivir | false | |
| 2-amino-4-chloro-3-hydroxybenzoic_acid | favipiravir | false | |
| 1_3-thiazole-4-carboxylic_acid | favipiravir | false | |
| 5-fluorocytosine | favipiravir | false | |
| 2_5-diaminopyrimidin-4_6-diol | favipiravir | false | |
| dabigatran_4-o-acylglucuronide_metabolite | remdesivir | false | |
| (3r_4s)-1-[(4-amino-5h-pyrrolo[3_2- | remdesivir | false | |
| d]pyrimidin-7-yl)methyl]-4- | | | |
| [(benzylsulfanyl)methyl]pyrrolidin-3-ol | | | |
| diodone | favipiravir | false | |
| methimazole_sulfinic_acid_metabolite_(ring) | favipiravir | false | |
| 2-amino-6-chloropyrazine | favipiravir | false | |
| didanosine | remdesivir | true | |
| fosifloxuridine_nafalbenamide | remdesivir | false | |
| 4-imino-5-methidyl-2- | favipiravir | false | |
| trifluoromethylpyrimidine | | | |
| pevonedistat | remdesivir | false | SARS-2 |
| remdesivir_nucleoside_triphosphate | favipiravir | false | SARS-2; |
| | | | MERS; |
| | | | SARS-1 |

Approved—drug is approved for human use;
CoV Inhibition—experimental evidence exists in the literature that the compound inhibits the indicated coronaviruses.

In one embodiment, the one or more antiviral compounds to be combined with coronavirus or administered to humans or animals infected with coronavirus is Cinacalcet, Alverine, Alvimopan, Antazoline, Bepridil, Darifenacin, Desipramine, Diphenhydramine, Diphenidol, Flunarizine, Fosphenytoin, Furazolidone, Imipramine, Lifitegrast, Masitinib, Mifepristone, Nicardipine, Nilotinib, Olopatadine, Ospemifene, Oxprenolol, Perhexiline, Phenoxybenzamine, Phenylbutazone, Phenyltoloxamine, Pizotifen, Promazine, Stiripentol, Tamsulosin, Zolmitriptan, and salts thereof or combinations thereof.

In another embodiment, the antiviral compound to be combined with coronavirus or administered to humans or animals infected with coronavirus is Cinacalcet. This compound is a secondary amine having the molecular formula C₂₂H₂₂F₃N. Cinacalcet is a commercially available prescription drug used to treat secondary hyperparathyroidism, parathyroid carcinoma, and primary hyperparathyroidism.

In another embodiment, the antiviral compound to be combined with coronavirus or administered to humans or animals infected with coronavirus is Alverine. This compound is a tertiary amine having one ethyl and two 3-phenylprop-1-yl groups attached to the nitrogen and has the molecular formula C₂₀H₂₇N. Alverine is a commercially available prescription drug used for functional gastrointestinal disorders.

In another embodiment, the antiviral compound to be combined with coronavirus or administered to humans or animals infected with coronavirus is Alvimopan. This compound has the molecular formula C₂₅H₃₂N₂O₄. Alvimopan is a peripherally acting μ opioid antagonist and is used to avoid postoperative ileus following small or large bowel restriction and accelerates the gastrointestinal recovery period.

In another embodiment, the antiviral compound to be combined with coronavirus or administered to humans or animals infected with coronavirus is Antazoline. This compound is a tertiary amine that has the molecular formula C₁₇H₁₉N₃. Antazoline is an ethylenediamine derivative with histamine H1 antagonist and sedative properties. Antazoline antagonizes histamine H1 receptors and prevents the typical allergic symptoms caused by histamine activities on capillaries, skin, mucous, membranes, and gastrointestinal and bronchial smooth muscles.

In another embodiment, the antiviral compound to be combined with coronavirus or administered to humans or animals infected with coronavirus is Bepridil. This compound is a tertiary amine in which the substituents on nitrogen are benzyl, phenyl and 3-(2-methylpropoxy)-2-(pyrrolidin-1-yl)propyl and has the molecular formula C₂₄H₃₄N₂O. Bepridil is an amine calcium channel blocker once used to treat angina. It is no longer sold in the United States, but may be commercially available elsewhere.

In another embodiment, the antiviral compound to be combined with coronavirus or administered to humans or animals infected with coronavirus is Darifenacin. This compound is an anticholinergic and antispasmodic agent and has the molecular formula C₂₈H₃₀N₂O₂. Darifenacin is a commercially available prescription drug used to treat urinary incontinence and overactive bladder syndrome.

In another embodiment, the antiviral compound to be combined with coronavirus or administered to humans or animals infected with coronavirus is Desipramine. This compound is an antidepressant and nerve pain medication and has the molecular formula C₁₈H₂₂N₂. Desipramine is a commercially available prescription drug used to treat depression.

In another embodiment, the antiviral compound to be combined with coronavirus or administered to humans or animals infected with coronavirus is Flunarizine. This compound is a selective calcium entry blocker with calmodulin binding properties and histamine H₁ blocking activity and has the molecular formula C₂₆H₂₆F₂N₂. Flunarizine is a commercially available prescription drug used to treat various indications such as migraines, occlusive peripheral vascular disease, vertigo and epilepsy. It is not available by prescription in the United States or Japan.

In another embodiment, the antiviral compound to be combined with coronavirus or administered to humans or animals infected with coronavirus is Fosphenytoin. This compound is a prodrug of phenytoin with the molecular formula C₁₆H₁₅N₂O₆P. Fosphenytoin is hydrolyzed to phenytoin by phosphatases. Phenytoin exerts its effect mainly by promoting sodium efflux and stabilizes neuronal membranes in the motor cortex. This leads to a suppression of excessive neuronal firing and limits the spread of seizure activity.

In another embodiment, the antiviral compound to be combined with coronavirus or administered to humans or animals infected with coronavirus is Furazolidone. This compound is a nitrofuran antimicrobial agent with the molecular formula C₈H₇N₃O₅. Furazolidone is used in the treatment of diarrhea or enteritis caused by bacteria or protozoan infections. Furazolidone is also active in treating typhoid fever, cholera and salmonella infections.

In another embodiment, the antiviral compound to be combined with coronavirus or administered to humans or animals infected with coronavirus is Imipramine. This compound is a tricyclic antidepressant with the molecular formula C₁₉N₂₄N₂. Imipramine is a synthetic tricyclic derivative, antidepressant that enhances monoamine neurotransmission in certain areas of the brain. Imipramine also induces sedation through histamine 1 receptor blockage, hypotension through beta-adrenergic blockage, and diverse parasympatholytic effects.

In another embodiment, the antiviral compound to be combined with coronavirus or administered to humans or animals infected with coronavirus is Lifitegrast. This compound is an N-acyl-L-alpha-amino acid and has the molecular formula C₂₉H₂₄C₁₂N₂O₇S. Lifitegrast is and FDA approved drug for the treatment of keratoconjunctivitis sicca (dry eye syndrome). Lifitegrast has a role as an anti-inflammatory drug and a lymphocyte function-associated antigen-1 antagonist.

In another embodiment, the antiviral compound to be combined with coronavirus or administered to humans or animals infected with coronavirus is Masitinib. This compound is a tyrosine-kinase inhibitor and has the molecular formula C₂₈H₃₀N₆OS. Masitinib is a commercially available prescription drug used to treat mast cell tumors in animals, specifically dogs and is commercially available in Europe.

In another embodiment, the antiviral compound to be combined with coronavirus or administered to humans or animals infected with coronavirus is Mifepristone. This compound is 3-oxo-Delta(4) steroid, an acetylenic compound and a tertiary amino compound and has the molecular formula C₂₉H₃₅NO₂. Mifepristone is a potent synthetic steroidal antiprogesterone which is used as a single dose in combination with misoprostol, a prostaglandin analogue, to induce medical abortion. Mifepristone alone, without misoprostol, is also approved as therapy of Cushing syndrome where it is given in a higher dose and for extended periods.

In another embodiment, the antiviral compound to be combined with coronavirus or administered to humans or animals infected with coronavirus is Nicardipine. This compound is a second generation calcium channel blocker and has the molecular formula C₂₆H₂₉N₃O₆. Nicardipine is used in the treatment of hypertension and stable angina pectoris.

In another embodiment, the antiviral compound to be combined with coronavirus or administered to humans or animals infected with coronavirus is Nilotinib. This compound is a selective tyrosine kinase receptor inhibitor and has the molecular formula C₂₈H₂₂F₃N₇O. Nilotinib is a commercially available prescription drug used to treat chronic myelogenous leukemia.

In another embodiment, the antiviral compound to be combined with coronavirus or administered to humans or animals infected with coronavirus is Olopatadine. This compound is a histamine-1 receptor inhibitor and has the molecular formula C₂₁H₂₃NO₃. Olopatadine stabilizes mast cells and prevents histamine release from mast cells. Also, Olopatadine blocks histamine H₁ receptors, preventing histamine from binding to the receptors.

In another embodiment, the antiviral compound to be combined with coronavirus or administered to humans or animals infected with coronavirus is Ospemifene. This compound is classified as a hormone and has the molecular formula C₂₄H₂₃ClO₂ Ospemifene is a commercially available prescription drug used to treat women with moderate to severe dyspareunia (painful intercourse) and moderate to severe vaginal dryness caused by menopause.

In another embodiment, the antiviral compound to be combined with coronavirus or administered to humans or animals infected with coronavirus is Oxprenolol. This compounds is an aromatic ether and has the molecular formula C₁₅H₂₃NO₃. Oxprenolol is a beta-adrenergic antagonist used in the treatment of hypertension, angina pectoris, arrhythmias, and anxiety.

In another embodiment, the antiviral compound to be combined with coronavirus or administered to humans or animals infected with coronavirus is Perhexiline. This compound is a member of piperidines and has the molecular formula C₁₉H₃₅N. Perhexiline has a role as a cardiovascular role. Perhexiline is a coronary vasodilator used especially for angina of effort.

In another embodiment, the antiviral compound to be combined with coronavirus or administered to humans or animals infected with coronavirus is Phenoxybenzamine. This compound is a synthetic, dibenzamine alpha adrenergic antagonist with antihypertensive and vasodilatory properties, classified as an alpha blocker, and has the molecular formula C₁₈H₂₂ClNO. Phenoxybenzamine is a commercially available prescription drug used to treat hypertension and carcinoid tumors.

In another embodiment, the antiviral compound to be combined with coronavirus or administered to humans or animals infected with coronavirus is Phenylbutazone. This compound is a nonsteroidal anti-inflammatory drug (NSAID) and has the molecular formula C₁₉H₂₀N₂O₂. Phenylbutazone is a commercially available prescription drug used to treat pain and fever in animals and is also used to treat ankylosing spondylitis in humans in the UK when other therapies are unsuitable.

In another embodiment, the antiviral compound to be combined with coronavirus or administered to humans or animals infected with coronavirus is Phenyltoloxamine. This compound is an ethanolamine derivative and has the molecular formula C₁₇H₂₁NO. Phenyltoloxamine has antihistaminic properties, it blocks H1 histamine receptors, thereby inhibiting phospholipase A2 and production of endothelium-derived relaxing factor, nitric oxide resulting in decreased cyclic GMP levels, thereby inhibiting smooth muscle constriction of various tissues, decreasing capillary permeability and decreasing other histamine-activated allergic reactions.

In another embodiment, the antiviral compound to be combined with coronavirus or administered to humans or animals infected with coronavirus is Pizotifen. This compound is a benzocycloheptathiophene and has the molecular formula C₁₉H₂₁NS. Pizotifen is a sedating antihistamine, with strong serotonin antagonist and weak antimuscarinic activity. Pizotifen is generally used as the malate salt for the treatment of migraine and the prevention of headache attacks during cluster periods.

In another embodiment, the antiviral compound to be combined with coronavirus or administered to humans or animals infected with coronavirus is Promazine. This compound is classified as a phenothiazine antipsychotic and has the molecular formula C₁₇H₂₀N₂S. Promazine is a commercially available drug used to lower blood pressure in animals suffering from laminitis and renal failure, and is also used as a pre-anesthetic agent to induce moderate sedation in animals.

In another embodiment, the antiviral compound to be combined with coronavirus or administered to humans or animals infected with coronavirus is Stiripentol. This compound is a phenylpropanoid and has a molecular formula C₁₄H₁₈O₃. Stiripentol is an anticonvulsant drug used as adjuvant therapy of Dravet syndrome, a rare form of severe childhood epilepsy.

In another embodiment, the antiviral compound to be combined with coronavirus or administered to humans or animals infected with coronavirus is Tamsulosin. This compound is a selective alpha-lA and alpha-1B adrenoceptor antagonist and has the molecular formula C₂₀H₂₈N₂O₅S. Tamsulosin is used in the therapy of benign prostatic hypertrophy.

In another embodiment, the antiviral compound to be combined with coronavirus or administered to humans or animals infected with coronavirus is Zolmitriptan. This compound is a member of the triptan class of 5-hydroxytryptamine (5-HT) receptor agonists and has a molecular formula of C₁₆H₂₁N₃O₂. Zolmitriptan selectively binds to and activates 5-HT 1B receptors expressed in intracranial arteries and 5-HT 1D receptors located on peripheral trigeminal sensory nerve terminals in the meninges and central terminals in brainstem sensory nuclei. Receptor binding results in constriction of cranial vessels, reduction of vessel pulsation and inhibition of nociceptive transmission, thereby providing relief of migraine headaches.

The coronaviruses to be inhibited, prevented or disrupted include all species of coronavirus, such as, but not limited to Human coronavirus 229E (HCoV-229E); Human coronavirus OC43 (HCoV-OC43); Severe acute respiratory syndrome coronavirus (SARS-CoV); Human coronavirus NL63 (HCoV-NL63, New Haven coronavirus); Human coronavirus HKU1; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019”. The methods are particularly suitable for the inhibition and treatment of the lethal coronaviruses causing Severe Acute Respiratory Syndrome (SARS), Middle East Respiratory Syndrome (MERS) and 2019 Novel Coronavirus (COVID-19).

The antiviral compounds described herein may be suitable for parenteral, oral, inhalation spray, topical, rectal, nasal, buccal, vaginal, or implanted reservoir administration. The term parenteral as used herein includes subcutaneous, intradermal, intravenous, intramuscular, intra-articular, intra-synovial, intrasternal, intrathecal, intrahepatic, intralesional and intracranial injection or infusion techniques. Optionally, the compounds described herein can administered orally, topically, intranasally, intravenously, subcutaneously, buccally, sublingually, intradermally, transdermally, intramucosally, intramuscularly, by inhalation spray, rectally, nasally, sublingually, buccally, vaginally or via an implanted reservoir.

In accordance with the methods provided herein, two or more of the compounds set forth above are combined in an antiviral composition or two or more of the compounds set forth above are administered sequentially.

The compounds described herein or derivatives thereof can be provided in one or more pharmaceutical compositions. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the one or more compounds described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected one or more compounds without causing unacceptable biological effects or interacting in a deleterious manner with the other components of the pharmaceutical composition in which it is contained.

The compositions can include one or more of the compounds described herein, one or more of the compounds described herein and one or more other active compounds or molecules. The composition can also include one or more of the compounds described herein, one or more of the compounds described herein and one or more other active compounds or molecules, and a pharmaceutically acceptable carrier. As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, N.J.), polyethylene glycol (PEG), and PLURONICS' (BASF; Florham Park, N.J.).

Compositions containing the one or more compounds described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release one or more of the active compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral or intravenous administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

The one or more compounds described herein, with or without additional agents, can be provided in the form of an inhaler or nebulizer for inhalation therapy. As used herein, inhalation therapy refers to the delivery of a therapeutic agent, such as the compounds described herein, in an aerosol form to the respiratory tract (i.e., pulmonary delivery). As used herein, the term aerosol refers to very fine liquid or solid particles carried by a propellant gas under pressure to a site of therapeutic application. When a pharmaceutical aerosol is employed, the aerosol contains the one or more compounds described herein, which can be dissolved, suspended, or emulsified in a mixture of a fluid carrier and a propellant. The aerosol can be in the form of a solution, suspension, emulsion, powder, or semi-solid preparation. In the case of a powder, no propellant gas is required when the device is a breath activated dry powder inhaler. Aerosols employed are intended for administration as fine, solid particles or as liquid mists via the respiratory tract of a patient.

The propellant of an aerosol package containing the one or more compounds described herein can be capable of developing pressure within the container to expel the one or more compounds when a valve on the aerosol package is opened. Various types of propellants can be utilized, such as fluorinated hydrocarbons (e.g., trichloromonofluromethane, dichlorodifiuoromethane, and dichlorotetrafluoroethane) and compressed gases (e.g., nitrogen, carbon dioxide, nitrous oxide, or Freon). The vapor pressure of the aerosol package can be determined by the propellant or propellants that are employed. By varying the proportion of each component propellant, any desired vapor pressure can be obtained within the limits of the vapor pressure of the individual propellants.

As described above, the one or more compounds described herein can be provided with a nebulizer, which is an instrument that generates very fine liquid particles of substantially uniform size in a gas. The liquid containing the one or more compounds described herein can be dispersed as droplets about 5 mm or less in diameter in the form of a mist. The small droplets can be carried by a current of air or oxygen through an outlet tube of the nebulizer. The resulting mist can penetrate into the respiratory tract of the patient.

Additional inhalants useful for delivery of the compounds described herein include intra-oral sprays, mists, metered dose inhalers, and dry powder generators (See Gonda, J. Pharm. Sci. 89:940-945, 2000, which is incorporated herein by reference in its entirety, at least, for inhalation delivery methods taught therein). For example, a powder composition containing the one or more compounds as described herein, with or without a lubricant, carrier, or propellant, can be administered to a patient. The delivery of the one or more compounds in powder form can be carried out with a conventional device for administering a powder pharmaceutical composition by inhalation.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the pharmaceutical compositions described herein can be substantially free from tetrazoles, triazoles, and/or solubility enhancers for monosodium urate. As used herein, the term “substantially free” from an indicated component (e.g., tetrazoles, triazoles, and/or solubility enhancers for monosodium urate), means that the pharmaceutical composition can include less than 1%, less than 0.1%, less than 0.01%, less than 0.001%, or less than 0.0001% of the component (e.g., a tetrazole, a triazole, and/or a solubility enhancer for monosodium urate) based on the weight of the pharmaceutical composition.

As noted above, the compositions can include one or more of the compounds described herein or pharmaceutically acceptable salts thereof. As used herein, the term pharmaceutically acceptable salt refers to those salts of the one or more compounds described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting one or more of the purified compounds in free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S.M. Barge et al., J. Pharm. Sci. (1977) 66, 1, which is incorporated herein by reference in its entirety, at least, for compositions taught therein.

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

The methods include administering to a subject an effective amount of one or more of the compounds or pharmaceutical compositions described herein, or a pharmaceutically acceptable salt thereof. The expression “effective amount,” when used to describe an amount of compound in a method, refers to the amount of a compound that achieves the desired pharmacological effect or other effect, for example, an amount that results in decreased viral load.

The compounds and compositions described herein or pharmaceutically acceptable salts thereof are useful for treating and/or preventing a disease or condition associated with coronavirus infection.

The methods described herein are useful for treating the diseases and conditions described herein in humans, including, without limitation, pediatric and geriatric populations, and in animals, e.g., veterinary application.

The following example will serve to further illustrate the present invention without, at the same time, however, constituting any limitation thereof. On the contrary, it is to be clearly understood that resort may be had to various embodiments, modifications and equivalents thereof which, after reading the description herein; may suggest themselves to those skilled in the art without departing from the spirit of the invention.

### Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

### Example 1: Coronavirus In Silico Drug Discovery Platform

Suitable coronavirus antiviral compounds were identified by extensively applied state-of-the-art computer codes by combining virus targets, in vitro and in vivo information about antiviral compounds in the coronavirus context and a wide range of libraries of compounds. Computational steps included:

The initial structures for all drug-candidates were collected from sources, as described above, and subsequently refined by optimizing geometries and electronic structures with several approaches, including quantum chemistry within density functional theory (DFT) methods and the suite of programs available in Maestro code by Schrödinger, LLC. The resulting refined structures were next implemented in blind docking calculations, an approach that allows to scan the whole protein surface in the search of main binding pockets.

Only the best poses were retained for the analysis, so that the provided structures correspond to the drug-target interaction with the largest affinity.

Large libraries of compounds have been systematically screened by using several computational approaches. More specifically, compounds are initially generated by accounting for physiological conditions. The resulting refined structures were next implemented in blind docking calculations, an approach that allows scanning of the whole protein surface in the search of main binding pockets. Only the top poses were retained for the analysis, so that the provided structures correspond to the drug-target interaction with the largest affinity.

All viral proteins for SARS-CoV-2 were obtained via curating solved protein crystal structures or high-confidence homology modelling of those proteins that do not have solved crystal structures. The resulting refined chemical compounds and viral protein structures were next implemented in the CANDO (Computational Analysis of Novel Drug Opportunities) platform to generate predicted binding affinities for every drug-candidate against every SARS-CoV-2 and human protein.

Predicted antiviral compounds are predicted via multiple routes. Known effective antivirals against MERS-CoV, SARS-CoV, or SARS-CoV-2 are used as positive controls to identify compounds within our library that have similar proteomic interaction or molecular fingerprint signatures. Candidate compounds with high similarity to multiple positive controls, for example, can have antiviral activity against SARS-CoV-2 (Table 2).

In addition, compounds that are identified to have high affinity for a specific target were predicted as SARS-CoV-2 antivirals (Table 1).

The results are summarized as follows: The 3C-like proteinase is a key target for blocking viral replication. The NSP3-papain proteinase is a key target for blocking viral replication. The RNA-directed RNA polymerase (RdRP) is a key target for blocking viral replication. The site consists of two adjacent aspartic acid residues. Compounds disrupt RdRP function when it binds and is potentially effective against at least two different coronaviruses. Compounds with high similarity to both remdesivir and favipiravir will bind the RdRP and inhibit viral replication (Table 3).

### Example 2: In vitro inhibitory activity of select compounds described herein against

SARS-CoV-2

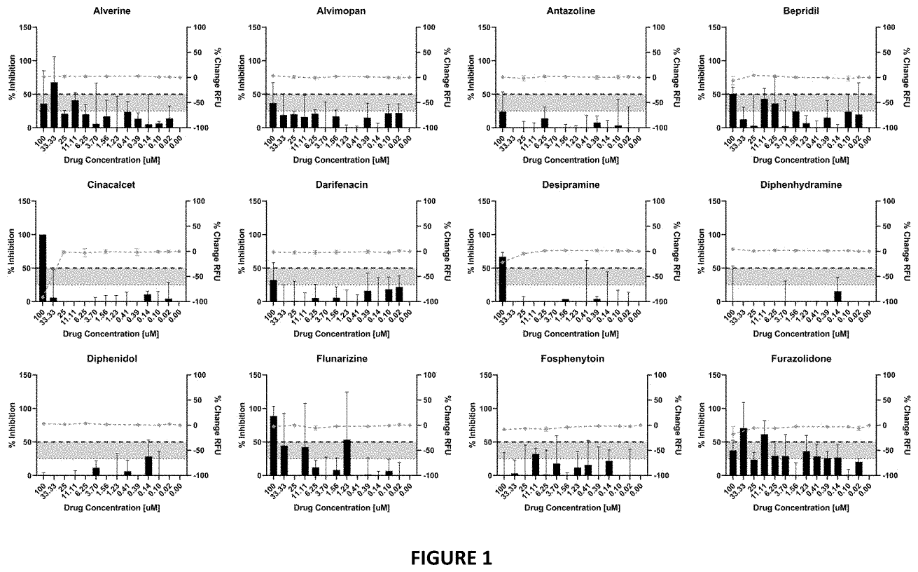

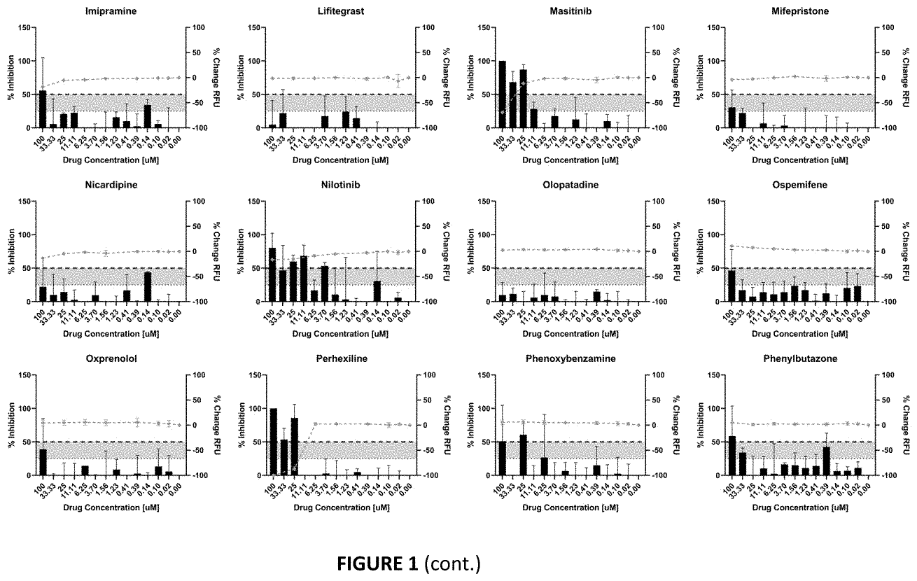

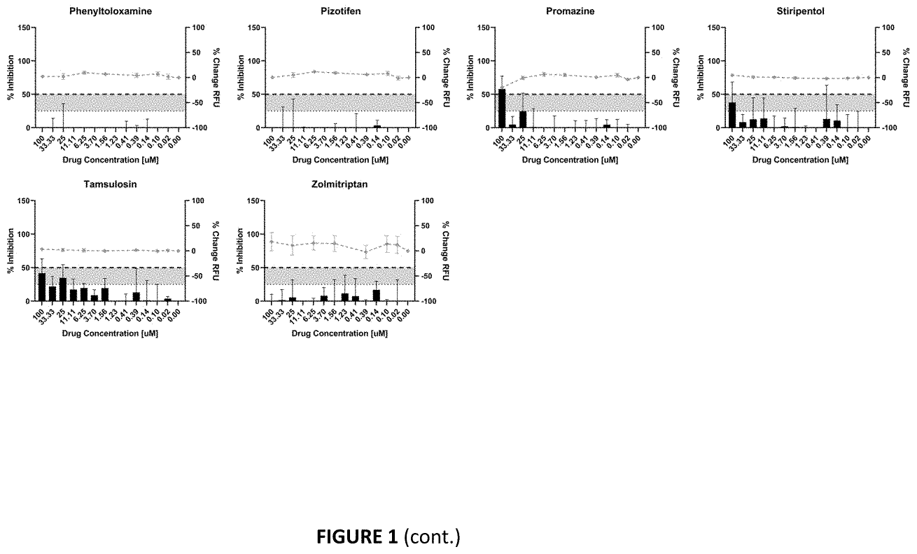

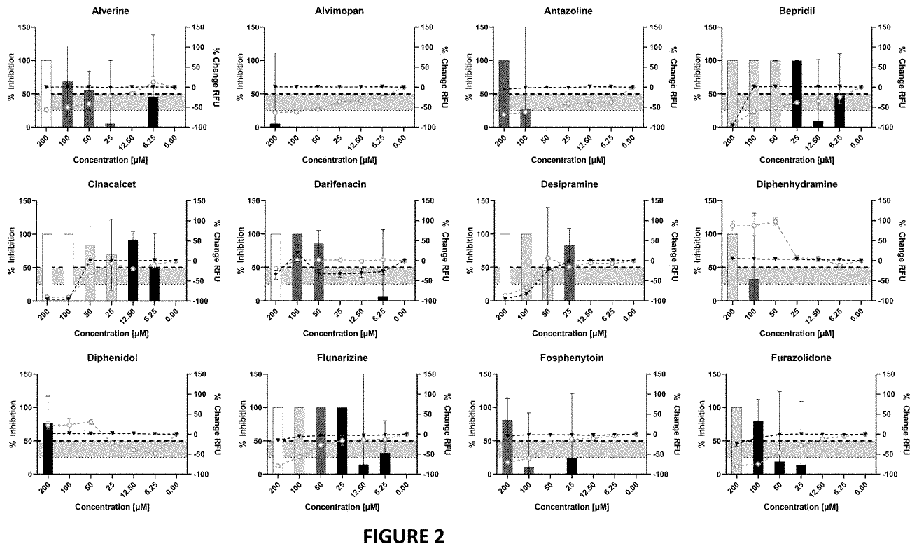

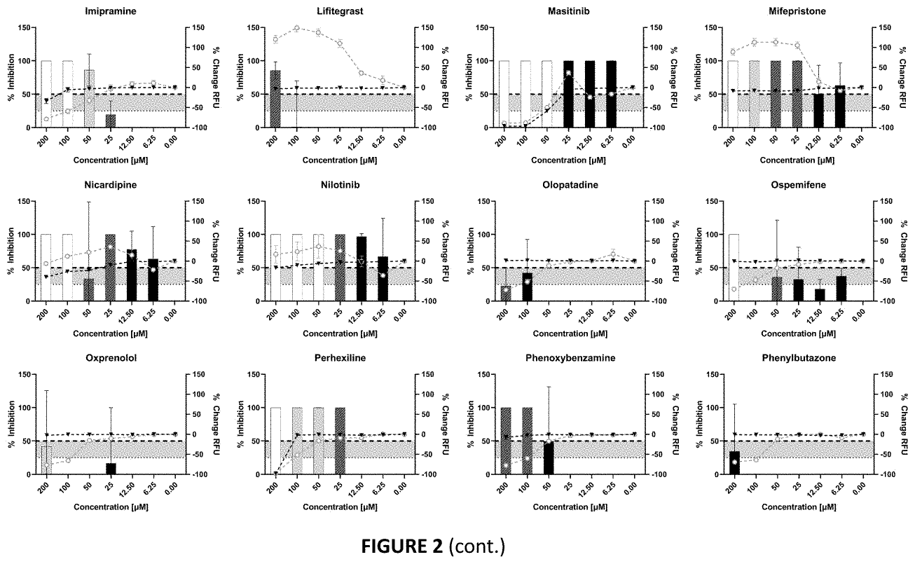

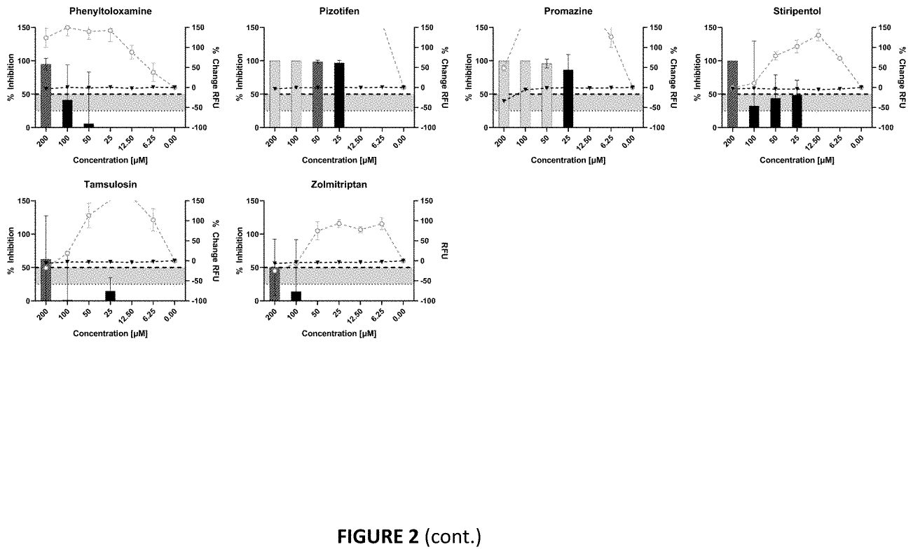

Select compounds specified herein and identified using the platform described in Example 1 were tested for toxicity and the ability to inhibit SARS-CoV-2 virus. The tested compounds, also referred to in this example as a drug or drugs, include the following: Cinacalcet, Alverine, Alvimopan, Antazoline, Bepridil, Darifenacin, Desipramine, Diphenhydramine, Diphenidol, Flunarizine, Fosphenytoin, Furazolidone, Imipramine, Lifitegrast, Masitinib, Mifepristone, Nicardipine, Nilotinib, Olopatadine, Ospemifene, Oxprenolol, Perhexiline, Phenoxybenzamine, Phenylbutazone, Phenyltoloxamine, Pizotifen, Promazine, Stiripentol, Tamsulosin, Zolmitriptan, and salts thereof or combinations thereof. The data show both entry and replication inhibition when pre-incubated with virus (FIG. 1) as well as when pre-incubated with cells and virus (FIG. 2). The data further show inhibitory activity at concentrations that are not toxic to cells in both FIG. 1 and FIG. 2.

### Methods

Compounds were tested for toxicity and their ability to inhibit SARS-CoV-2 infection and replication in a tissue culture system. Master stock solutions of each compound was prepared at 10 mM in DMSO and stored at 4° C. in the dark.

A first inhibition assay (“One-Step Assay”) focused on measuring inhibition caused by compound interaction with the viral particle during entry and infection, while a second inhibition assay (“Two-Step Assay”) focused on measuring inhibition caused by interactions with viral and cellular targets during the viral replication and progeny production processes. A cellular metabolism assay was performed for all compound concentrations using the Cell Titer Blue™ kit sold by Promega Corp. (Madison, Wis.) to verify that observed viral inhibition was not due to toxicity or a reduction in cellular metabolism induced by the compounds.

The One-Step Assay was designed to evaluate the ability of a compound to inhibit entry of virus into the cell. It was performed by allowing varying compound concentrations to incubate with a sample of infectious SARS-CoV-2 virus particles for 1 hour at 37° C. in the dark. Following, the virus/compound mixture was allowed to infect a monolayer of susceptible Vero E6 African green monkey kidney cells. After 15 minutes, methylcellulose overlay was added to prevent virus particle diffusion. The infected cell monolayers were allowed to grow at 37° C. for 24 hours at which point the cell monolayers were fixed and stained for SARS-CoV-2 antigens using immunohistochemistry using a monoclonal antibody specific for the SARS-CoV-2 spike protein. The resulting number of stained viral foci was used to quantify infection inhibition. Results are summarized in FIG. 1 and methods for one step assay used for FIG. 1 follow:

### One-Step General Methods:

### Compound Stocks and Dilutions

A master stock of each compound (10 mM) was generated in 100% DMSO (Sigma, D8418) and stored at 4° C., in the dark. On the day of each experiment, master stocks of each compound were diluted to a working stock of 200 μM by adding 3 μL master stock into 147 μL of Viral Transport media (VTM). Serial dilutions (3- or 4-fold) were then performed in VTM. Virus was added to each dilution as described below.

### Antigen Propagation and Dilution

SARS-CoV-2 virus (USA-WA1/2020, BEI Resources, NR-52281; SARS-2) was propagated in Vero E6 cells to passage 3 (P3) and subsequently tittered at 8.0×10⁶ FFU/mL. Stock virus was diluted by transferring 30 μL into 40 mL VTM. Next, 10 μL, (equivalent of 50 FFU) of this working stock of virus was transferred onto all wells of the drug dilution plate (see section above) except for the negative control wells (run in triplicate) and incubated for 1 hours at 37° C., in the dark.

### Focus Forming Units (FFU) Assay

Plates containing virus were incubated at 37° C. for 15 minutes and 60μ1 of overlay media (OptiMEM containing 0.8% methylcellulose, 2% FBS and 1×A/A) was added. After 24 hours of incubation at 37° C., overlay was decanted, and plates were washed three times with 1×PBS. Fixative solution (80% methanol, 20% acetone), was added at 100 μl/well and incubated a minimum of 10 minutes at room temperature before submersion in fixative solution for removal from BSL-3 facilities. Next, 50 μL/well of primary antibody specific for SARS-CoV-2 spike protein (HRP-conjugated 1CO2, 1.4 μg/ml) diluted 1:1000 in blocking buffer (0.1% PBS-Tween20 (PBS-T), 5% NFDM) was incubated at room temperature for 1 hour. Primary antibody was decanted, monolayers washed three times using 0.1% PBS-T followed by one wash with dH2O. Finally, 75 μL/well 3,3′,5,5′-tetramethylbenzidine (TMB) development solution with stabilizer was added and incubated at room temperature for 20 minutes. Plates were rinsed under tap water and allowed to air dry completely. Imaging for manual FFU quantification was obtained using a BioTek Cytation7 imager.

The Two-Step Assay evaluated the overall ability of a compound to inhibit entry and replication of the virus in the cells. For this assay, dilutions of the compounds were pre-incubated with virus-susceptible cell monolayers overnight. Virus was then exposed separately to the same compound dilutions and incubated for one hour. Media was removed from the compound-treated cells and replaced with the virus-compound dilutions. These cells were allowed to incubate at 37° C. for 48 hours. The viral titer of each well was then tested by FFU assay as described, to determine the degree of inhibition by each compound. Percent inhibition was determined as compared to control (which is the virus and media only with no compound). Results are summarized in FIG. 2.

### Toxicity

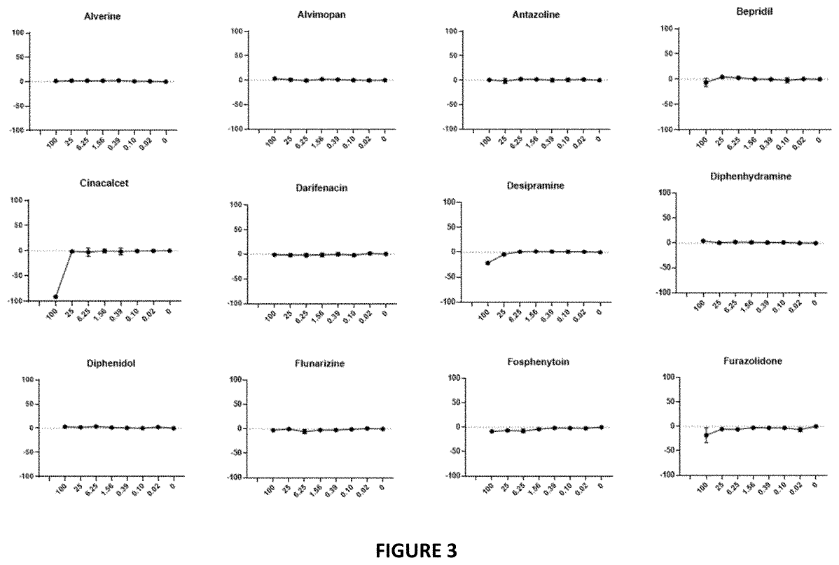

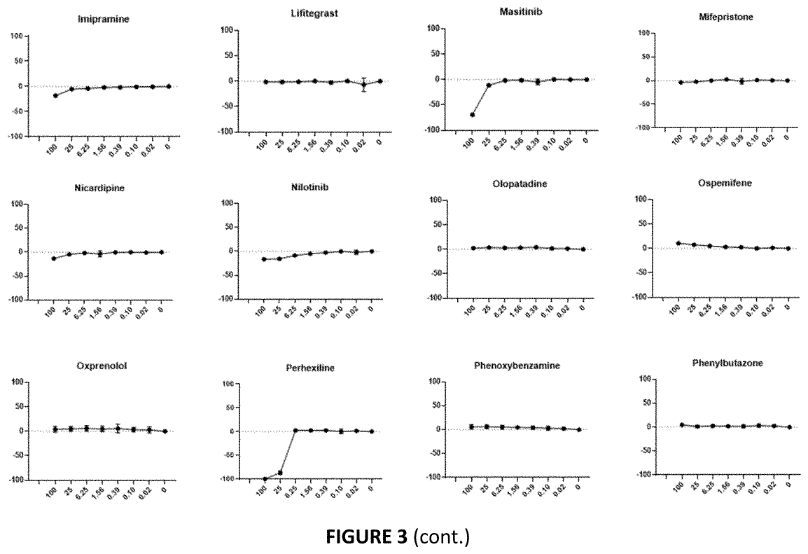

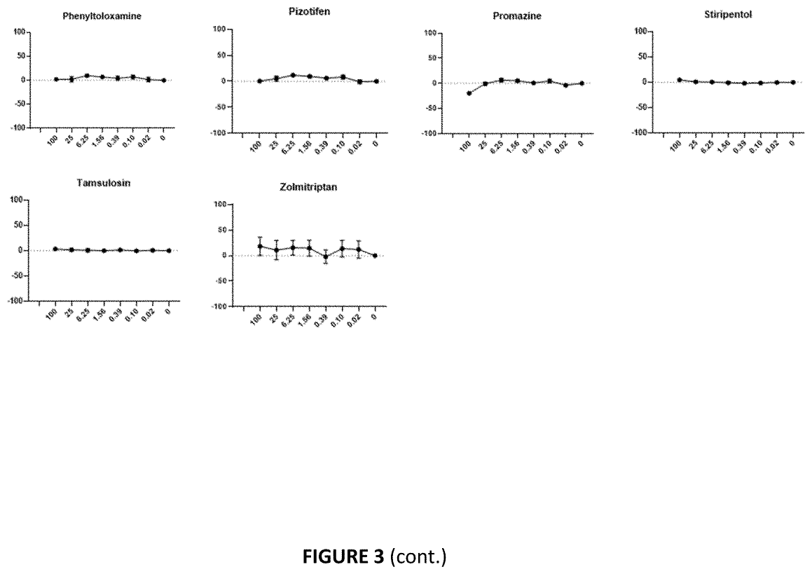

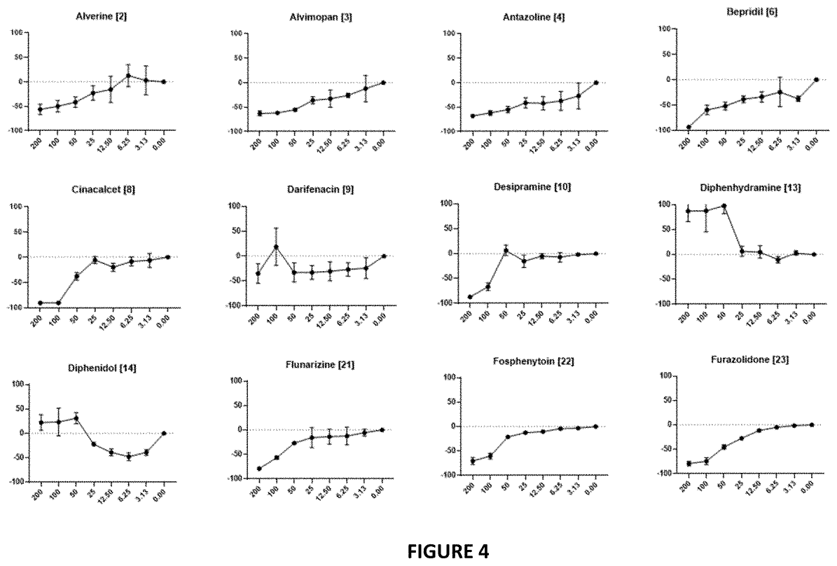

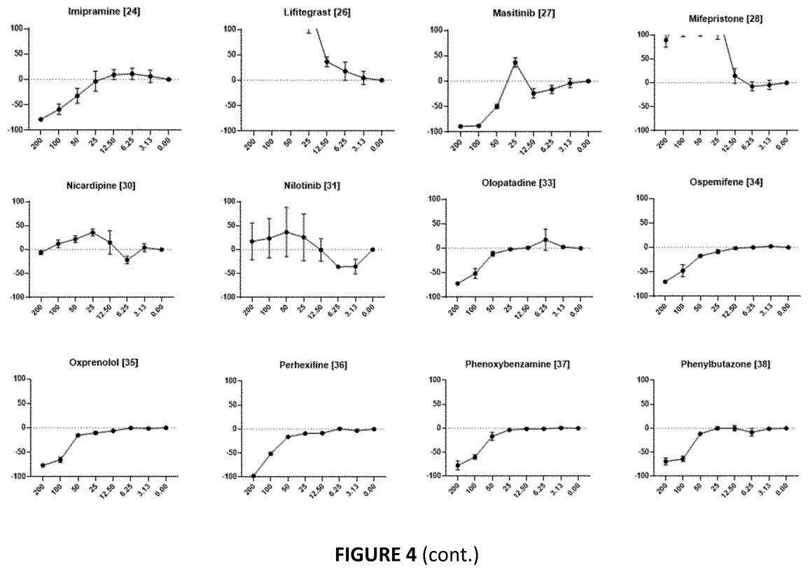

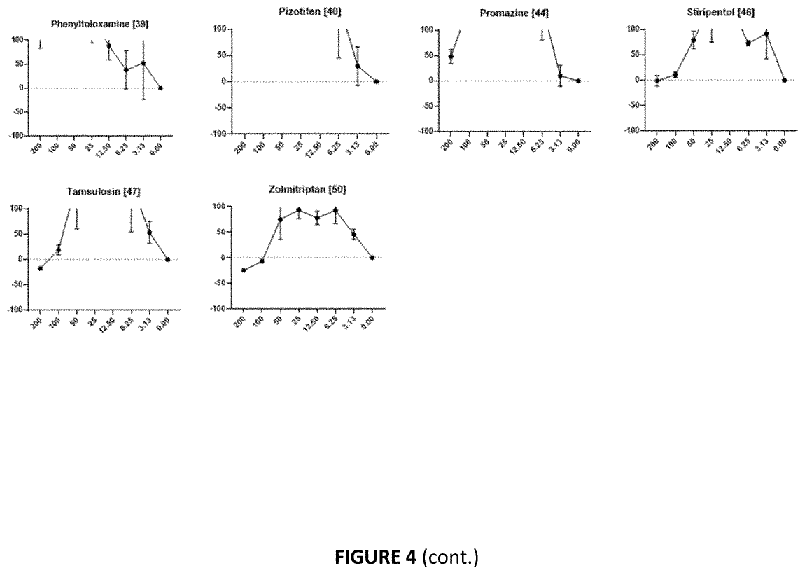

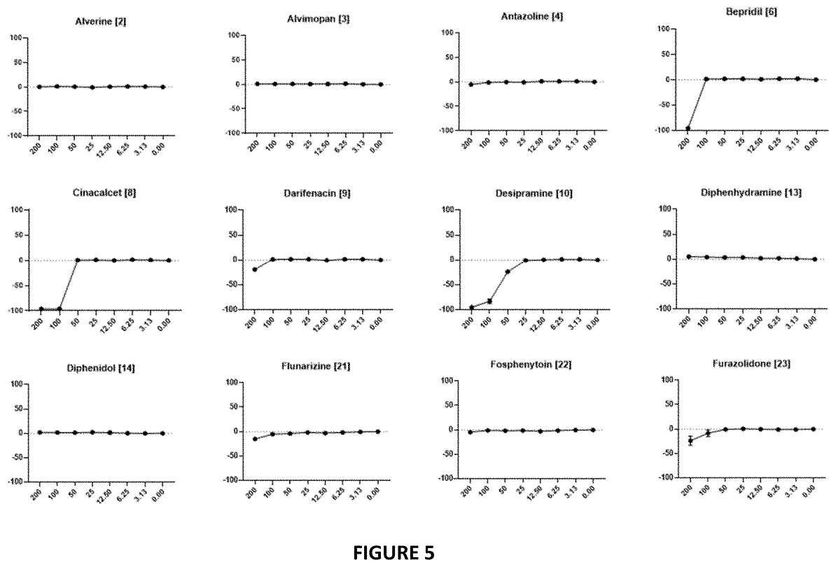

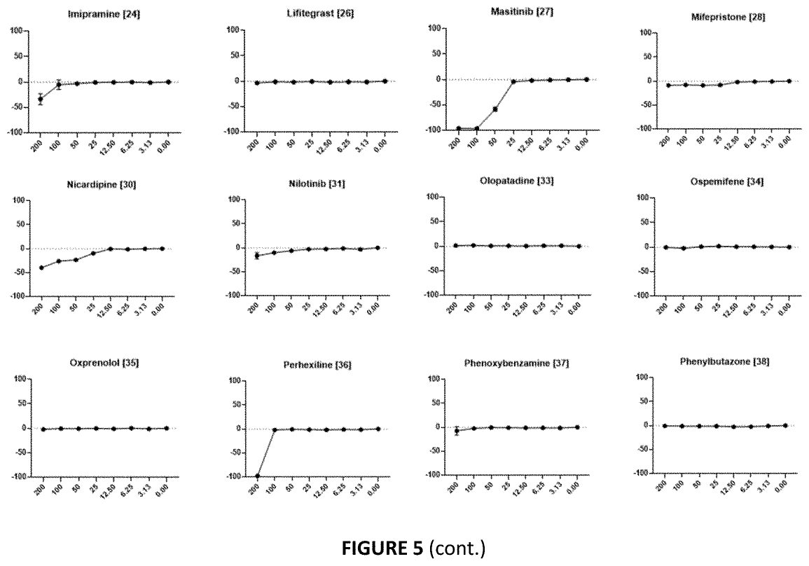

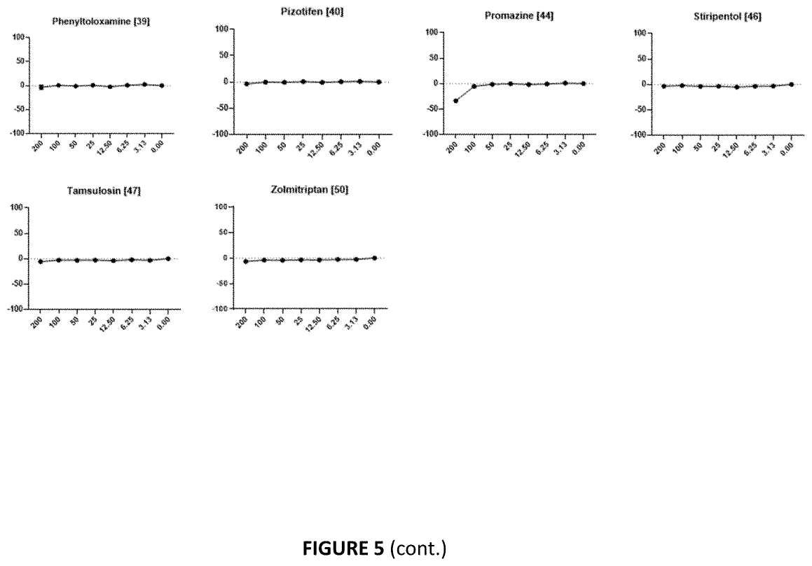

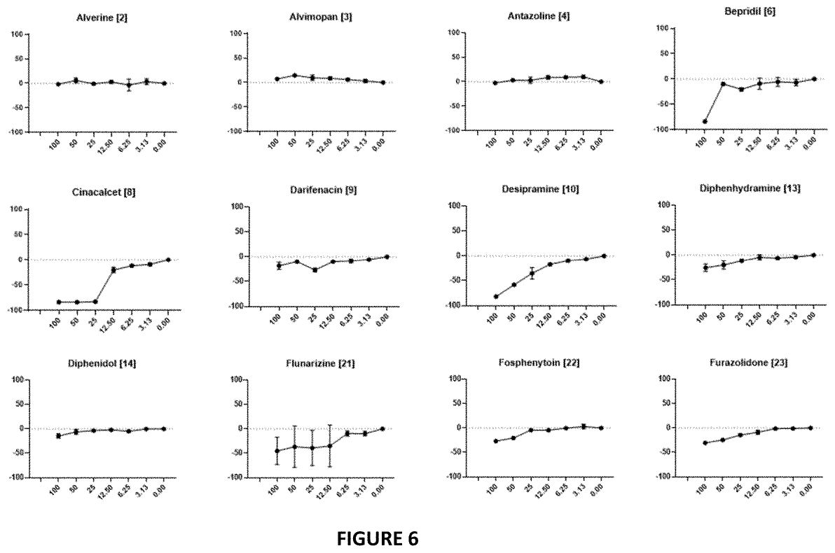

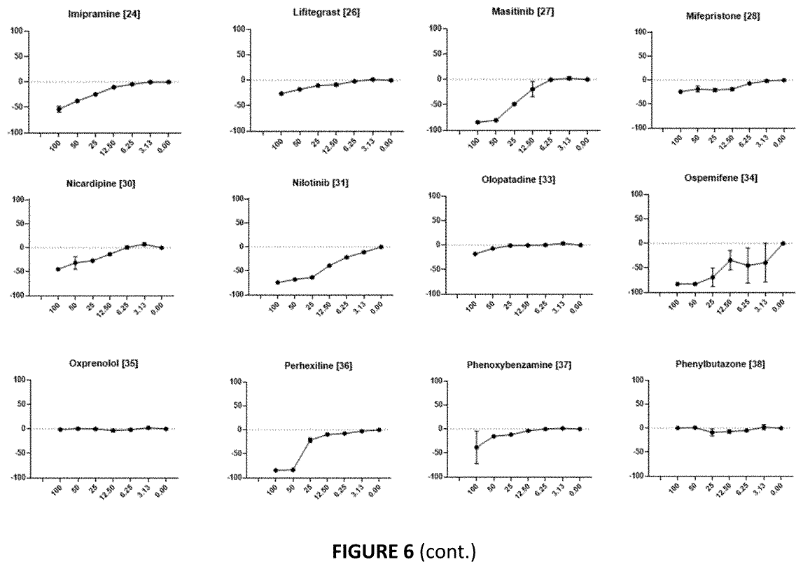

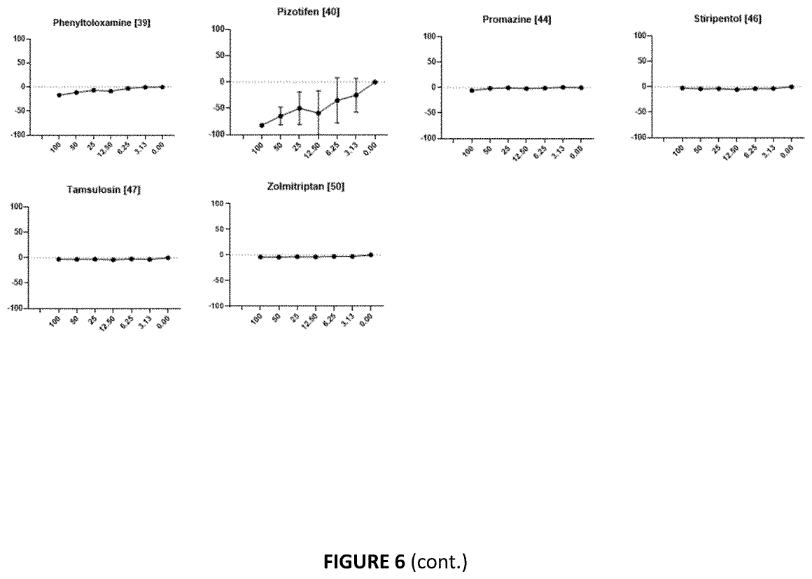

Evaluation of individual drug toxicity was measured using CellTiter-Blue® Cell Viability Assay G8081 (Promega Corp., Madison, Wis.) with a fluorometric readout. Vero E6 African green monkey kidney cells were propagated in growth media of Dulbecco's Modified Eagle Medium (DMEM) containing 10% Fetal Bovine Serum (FBS) and 1×antibiotic/antimycotic (A/A). The day before each assay, 3-4.0×10³ cells were seeded in 100 culture media per well in 384 well cell culture microplates (Cellstar, 781091; CellStar Corporation, Dallas, Tex.) and incubated overnight at 37° C., 5% CO₂ with humidity in 384 Cell culture microplates (Cellstar, 781091). Following establishment of a confluent monolayer, growth media was decanted, and cells exposed to each compound for 20-22 hours at 37° C., 5% CO2 in the dark. The compounds as described herein were added in a 50 μL final volume of viral transport media (DMEM+2% FBS+1× A/A) ranging from 0.02-200 μM based on individual assay design. After initial exposure, CellTiter Blue® reagent was added. Cells were returned to incubate under standard cell culture conditions for at least four hours. Results were recorded by measuring fluorescence at 560_{Ex}/590_{Em} 24-48 hours post drug exposure. Percent change in Relative Fluorescent Units (RFU) was calculated from baseline media-only control wells for each compound. Additional controls included no drug (media only), 10% DMSO and hydroxychloroquine (HXQ) at comparable concentrations. Results having different time points and concentrations are summarized in FIG. 3 through FIG. 6.

Cellular Pre-Incubation Assay: Two-Step Assay Exemplary Methods Exemplary Drug Dilution

The drug was diluted to 200 μM by adding 3 μL of the drug stock into 147 of Viral Transport media (VTM) which consists of Dulbecco's Modified Eagle Medium (DMEM) with 2% fetal bovine serum (FBS) and 1× of Antibiotic/Antimycotic (A/A). As noted above, 3-fold serial dilutions took place by transferring 50 μL of the 200 μM compound into 100 μL of viral transport media (VTM) in seven steps keeping the final row free of drug.

Two 384 well plates were seeded with 3000-4000 Vero E6 cells per well. Once monolayers were confluent, drug dilutions were made in VTM. Further, 200 μM dilutions of drug were made by adding 3 μL of 10 mM stock into 147 μL of VTM. Serial dilutions were performed in VTM. Growth media was removed from Vero E6 cells and 30 μL of the drug dilutions were added in each well. The remaining volume of the drug dilution was saved at 4° C. overnight. Cells were incubated overnight at 37° C.

On the second day in Bio-Safety Level 3 (BSL3), virus was diluted by adding 10 μL of P3 SARS-CoV-2 stock to 10 mL of VTM. Then, 5 μL of diluted virus was added to the remaining volume of the drug dilutions with a target of 0.01 multiplicity of infection (MOI). The mixture was incubated for 1 hour at 37° C. The 384 well plates that had been pre-treated with drug overnight were then dumped and 75 μL of the virus/drug dilutions was added such that the drug concentrations remained the same in each well. The resulting mixture was incubated for 48 hours.

On day three, 384 well plates were seeded with Vero E6 at 3000 cells per well. Four plates were used for each series of 16 treatments.

On day four, the Focus Forming Unit (FFU) assay was used to determine titer of the supernatant of each of the wells. For the assay, each infected well was sampled and serially diluted. Vero E6 growth media was removed from the fresh monolayers of the FFU plates prepared on day 3 and 20 μL of media from the serial dilutions of the virus infected well was added in triplicate. The FFU plate was allowed to incubate at 37° C. for 20 minutes. Following, 60 μL of methyl-cellulose was added and incubated at 37° C. for 24 hours.

The next day, the overlay was washed from the FFU wells by repeated gentle spray or dip in PBS. Moisture was removed and the cells were fixed in methanol/acetone (80% and 20%). Plates were then removed from BSL3 and were ready for immunostaining for FFU.

### Viral Preparation

SARS-CoV-2 virus (USA-WA1/2020, BEI Resources, NR-52281; SARS-2) was propagated in Vero E6 cells. Passage 3 (P3) containing 8.0×10⁶ FFU/mL was diluted by transferring 30 μL of the virus into 40 mL VTM. Next, 10 μL (equivalent of 50 FFU) of diluted virus was transferred onto all wells of the drug dilution plate except for the negative control wells (run in triplicate) and incubated for 1 hour at 37° C., in the dark.

### Results

In an experiment using the Two-Step Assay, cells were pre-exposed overnight to the select compounds for 24 hours. FIG. 2 shows the FFU count overlay following infection highlighting percent inhibition with visual toxicity data. The left y-axis shows the percent inhibition of SARS-CoV-2 from three repeats of the Two-Step Assay. Phenotypic observations are illustrated by changes in bar color at each concentration. The white bars of FIG. 2 represent a phenotype showing monolayer loss (complete or partial) in three out of three repeat assays. Gray bars of FIG. 2 represent a phenotype showing monolayer loss (complete or incomplete) in two out of three repeat assays. Gray striped bars of FIG. 2 represent a phenotype showing monolayer loss (complete or portion) in one out of three repeat assays. The black bars of FIG. 2 represent a phenotype showing no visual monolayer disturbance during the inhibition assay. In contrast, the right y-axis shows the percent change of Relative Fluorescent Units (RFU), representing cell viability. The blackdotted lines are plotted data collected 24 hour post-exposure to drug and 3-4 hours post-addition of Cell Blue viability reagent. The gray dotted lines are plotted data collected 48 hours post-exposure to drug and about 22 hours post-addition of Cell Blue viability reagent. Toxicity assays were performed at Biosafety Level 2 (BSL2) level. Following application of the different selected compounds to cells, the supernatant remained for the duration of the experiment (whereas when performing the inhibition assay protocol described above, the supernatant was removed after overnight treatment of the cells with the compound). Thus, removal of the supernatant may have led to loss of cells or prevention from recovery after the initial result.

Based on the toxicity and viral entry inhibition, the following compounds were identified as SARS-CoV-2 virus inhibitors highly suitable for use as therapeutic agents: Cinacalcet, Masitinib, Phenoxybenzamine, Phenylbutazone and Flunarizine. Additional compounds that are suitable for use as therapeutic agents are listed in Table 4, which provides information regarding the percent inhibition at the highest non-toxic concentration, toxicity and repeatable result.

TABLE 4
COVID-19 Entry Inhibition In Vitro Data

| | % Inhibition | | |
|---|---|---|---|
| | at HIGHEST | | |
| | Non-toxic | TOXICITY | REPEATABLE |
| COMPOUND | Concentration | Y/N | RESULT Y/N? |
| Alverine | 30% | N | N |
| Bepridil | 60% | N | Y |
| Darifenacin | 45% | N | Y |
| Desipramine | 60% | Very weak | Y |
| | | toxicity | |
| Flunarizine | 80% | N | Y |
| Masitinib | 90% | Y | Y |
| Nilotinib | 60% | N | Y |
| Ospemifene | 45% | N | Y |
| Phenoxybenzamine | 95% | N | N |
| Phenylbutazone | 90% | N | N |
| Promazine | 70% | Very weak | Y |
| | | toxicity | |

All patents, publications and abstracts cited above are incorporated herein by reference in their entirety. It should be understood that the foregoing relates only to preferred embodiments of the present invention and that numerous modifications or alteration may be made therein without departing from the spirit and the scope of the present invention as defined in the following claims.

1. A method for treating or preventing coronavirus infection comprising:
administering to a human or animal infected with coronavirus, an effective amount of one or more antiviral compounds,
wherein the one or more antiviral compounds disrupts replication of the coronavirus; and
wherein the one or more antiviral compounds is selected from the group consisting of Cinacalcet, Alverine, Alvimopan, Antazoline, Bepridil, Darifenacin, Desipramine, Diphenhydramine, Diphenidol, Flunarizine, Fosphenytoin, Furazolidone, Imipramine, Lifitegrast, Masitinib, Mifepristone, Nicardipine, Nilotinib, Olopatadine, Ospemifene, Oxprenolol, Perhexiline, Phenoxybenzamine, Phenylbutazone, Phenyltoloxamine, Pizotifen, Promazine, Stiripentol, Tamsulosin, and Zolmitriptan, salts thereof or combinations thereof.

2. The method of claim 1, wherein the coronavirus is SARS-CoV-2.

3. The method of claim 1, wherein the one or more antiviral compounds inhibit function of coronavirus NSP3-papain proteinase.

4. The method of claim 3, wherein the one or more antiviral compounds bind to the coronavirus NSP3-papain proteinase.

5. The method of claim 1, wherein the one or more antiviral compounds inhibit function of coronavirus 3C-like proteinase.

6. The method of claim 5, wherein the one or more antiviral compounds bind to the coronavirus 3C-like proteinase.

7. The method of claim 1, wherein the one or more antiviral compounds inhibit function of coronavirus RNA-directed RNA polymerase (RdRP).

8. The method of claim 1, wherein the one or more antiviral compounds is administered to the human or animal by intravenous administration.

9. The method of claim 1, wherein the one or more antiviral compounds is administered to the human or animal by oral administration.

10. The method of claim 1, wherein the one or more antiviral compounds is administered to the human or animal by intranasal administration.

11. The method of claim 1, wherein the one or more antiviral compounds is administered to the human or animal by inhalation administration.

12. The method of claim 1, wherein the one or more antiviral compounds is administered to the human or animal by intracavity administration.

13. The method of claim 1, wherein the one or more antiviral compounds is administered to the human or animal by subcutaneous administration.

14. The method of claim 1, wherein the one or more antiviral compounds is administered to the human or animal by sublingual administration.

15. The method of claim 1, wherein the one or more antiviral compounds is administered to the human or animal by buccal administration.

16. The method of claim 1, wherein the one or more antiviral compounds is Cinacalcet.

17. The method of claim 1, wherein the one or more antiviral compounds is selected from the group consisting of Cinacalcet, Flunarizine, Masitinib, Phenoxybenzamine and Phenylbutazone, salts thereof or combinations thereof.

18. The method of claim 1, wherein the one or more antiviral compounds is administered in combination with a pharmaceutically acceptable carrier.
